(19) United States
(12) Patent Application Publication
(10) Pub. No.: US 2024/0230628 A1
(43) Pub. Date: Jul. 11, 2024

(54) HUMAN ENGINEERED CARDIAC TISSUE PLATFORM

(71) Applicant: The Trustees of Columbia University in the City of New York, New York, NY (US)

(72) Inventors: Gordana VUNJAK-NOVAKOVIC, New York, NY (US); Sharon FLEISHER, New York, NY (US); Manuel TAMARGO, Miami, FL (US); Trevor Ray NASH, New York, NY (US); Robert WINCHESTER, New York, NY (US); Laura GERALDINO-PARDILLA, Ardsley, NY (US)

(21) Appl. No.: 18/431,269

(22) Filed: Feb. 2, 2024

Related U.S. Application Data
(63) Continuation of application No. PCT/US2022/039146, filed on Aug. 2, 2022.
(60) Provisional application No. 63/228,581, filed on Aug. 2, 2021.

(51) Int. Cl. G01N 33/50; C12M 1/12; C12M 3/00; C12N 5/071

(52) U.S. Cl. CPC G01N 33/5082; C12M 21/08; C12M 25/14; C12N 5/0697; C12N 2513/00; C12N 2533/54; C12N 2533/56; C12N 2537/10; G01N 2800/32

(57) ABSTRACT

Provided are a system and method to culture human cardiac muscle tissues in a bioreactor comprising a plurality of culture wells arranged linearly between two carbon electrodes that are exposed within each well; and two horizontal parallel flexible pillars extending from each well wherein the pillars in each well are configured to suspend an engineered tissue. Also provided is method for diagnosing myocarditis in a subject by analyzing human cardiac muscle tissues cultured in the bioreactor in the presence of blood serum or antibodies from the subject.

### CROSS REFERENCE TO RELATED APPLICATION

This application is a continuation of PCT International Application No. PCT/US2022/039146, filed Aug. 2, 2022, which claims the benefit of U.S. Provisional Application No. 63/228,581, filed Aug. 2, 2021, the contents of which are incorporated herein in their entirety.

### STATEMENT OF GOVERNMENT INTEREST

This invention was made with government support under grants EB025765, EB027062 and HL076485 awarded by the National Institutes of Health. The U.S. government has certain rights in the invention.

### BACKGROUND

Numerous models of the human heart have been developed in the past few years, with the goal to generate minimally functional units that could capture the heart's (patho)physiology in a simplified manner. 2D monolayer cultures represent the most simple and high throughput model; however they lack the cell maturity required for most studies. 3D microspheres can overcome this limitation while enabling high throughput assays, but they lack scaffolding and mechanical cues, limiting their organization. Importantly, they do not allow the direct measurement of contraction force, a critical aspect in disease modeling studies. Although lower in throughput, 3D cardiac tissues represent a promising alternative for these models.

Cardiac tissues have been created through cell-gel compaction and cast around anchoring structures such as wires, posts and pillars to provide mechanical load, supporting tissue maturation. Further maturation can be achieved by providing constant electrical stimulation, mimicking the electrical pacing of cells in the native heart.

Major advances in stem cell biology and tissue engineering have enabled the generation of models of the adult human heart, or “hearts-on-a-chip” that are currently being used for various applications such as disease modeling, and drug development. While these methodologies to enhance cardiac tissue performance have been well established and harnessed by several groups, they are complicated to set up and require specialized apparatus. Beyond the varied bioreactors used by different groups, electrical stimulation equipment is also varied. Electrical stimulation equipment can be set to create variable electric fields, but most importantly vary in the max available charge injected, leading to difficulties in translating technologies between labs. With increasing interest in harnessing these models for basic and applied research, there is a need for a simple, and accessible methodology for the generation of engineered cardiac tissues and their assessment that could be adopted by any standard research laboratory.

Patients with systemic lupus erythematosus (SLE) and Rheumatoid Arthritis (RA) have rates of heart failure and associated mortality that are 2-3-fold higher than in the general population. This excess risk is evident after adjusting for traditional cardiovascular risk factors, suggesting that the adaptive autoimmune response intrinsic to these autoimmune diseases is the main driver. Autopsy studies show that 50-80% of SLE patients have myocarditis, likely subclinical, hence most of these cases remain undiagnosed and could contribute the overall cardiovascular mortality in these patients. Endomyocardial biopsies for direct examination of patient cardiac tissue are the current gold standard for diagnosis of myocarditis, however they are invasive and inaccurate. PET-CT to examine FDG uptake (indicative of active myocardial inflammation) can inform a clinician on whether a patient has myocarditis, however they are expensive and involve high levels of radiation. Therefore, there is a critical need to develop new strategies to diagnose subclinical lupus myocarditis.

### SUMMARY

To address these needs, we have developed a user-friendly, accessible, and customizable platform that will provide all the resources required for the fabrication of tissues, and which enable electromechanical stimulation, and real-time data acquisition and analysis.

In one aspect, provided is a system to culture human cardiac muscle tissues (such as millimeter-sized) comprising a bioreactor comprising a plurality of culture wells arranged linearly between two carbon electrodes that are exposed within each well; and two horizontal parallel flexible pillars extending from each well wherein the pillars in each well are configured to suspend an engineered tissue.

Embodiments of the system include the following, alone or in any combination.

The system further comprises a double bi-layer capacitor comprising apart carbon rods for reproducibly injecting a safe amount of charge that is compatible with cardiac tissue stimulation.

The system further comprises circuitry and software to provide cyclic electromechanical stimulation to the cardiac muscle tissues.

The system further comprises a microscope and software to control and synchronize video acquisition, electrical stimulation, and stage positioning.

The system wherein brightfield imaging and calcium imaging are acquired.

The engineered human cardiac muscle tissues are generated from induced pluripotent stem cell (iPSC)-derived cardiomyocytes, human dermal fibroblasts, cardiac fibroblast, and tissues from iPS-cardionyocytes and iPS-cardiac fibroblasts.

The engineered human cardiac muscle tissues comprise a collagen or fibrin hydrogel.

The tissues are derived from induced pluripotent stem cell (iPSC)-derived cardiomyocytes and cardiac fibroblasts within a fibrin hydrogel.

In another aspect, provided is a method for culturing engineered human cardiac muscle tissues, the method comprising disposing the tissues between the two horizontal parallel flexible pillars of the reactor described above; and providing cyclic electromechanical stimulation to the tissues.

Embodiments of the method include the following, alone or in any combination.

The engineered human cardiac muscle tissues are generated from induced pluripotent stem cell (iPSC)-derived cardiomyocytes, human dermal fibroblasts, cardiac fibroblast, and tissues from iPS-cardionyocytes and iPS-cardiac fibroblasts.

The engineered human cardiac muscle tissues comprise a collagen or fibrin hydrogel.

The tissues are derived from induced pluripotent stem cell (iPSC)-derived cardiomyocytes and cardiac fibroblasts within a fibrin hydrogel.

In another aspect, also provided is a method for diagnosing myocarditis in a subject, the method comprising culturing engineered cardiac muscle tissues in the system described above; adding the subject's blood serum or isolated antibodies to the bioreactor; culturing the engineered cardiac muscle tissues in the presence of the blood serum or antibodies for a period of time; measuring calcium handling and force generation of the engineered cardiac muscle tissue or immunostaining the tissues; and determining whether the measured calcium handling or force generation or immunostaining indicate that the subject is suffering from myocarditis.

Embodiments of the method include the following, alone or in any combination.

The method wherein culturing engineered cardiac muscle tissues comprises disposing the tissues between the two horizontal parallel flexible pillars of the reactor; and providing cyclic electromechanical stimulation to the tissues.

The subject is suffering from systemic lupus erythematosus or rheumatoid arthritis.

The engineered human cardiac muscle tissues are derived from induced pluripotent stem cell (iPSC)-derived cardiomyocytes and cardiac fibroblasts within a fibrin hydrogel.

The engineered human cardiac muscle tissues are co-cultured with autoantibodies from subjects' sera.

The method wherein ¹⁸F-fluorodeoxyglucose-positron emission tomography/computed tomography (¹⁸F-FDG-PET/CT) is used to quantify extent of myocardial inflammation.

The method comprising culturing the engineered human cardiac muscle tissues in a medium to shift cell metabolism from anaerobic glycolysis toward fatty acid oxidation; and subjecting the engineered human cardiac muscle tissues to electrical stimulation to induce macroscopic contractions.

### BRIEF DESCRIPTION OF THE DRAWINGS

FIG. 1 shows aspects of a platform for culturing engineered human cardiac muscle tissue, according to an exemplary embodiment of the disclosed subject matter.

FIGS. 2A-2E show aspects of reactor design and characterization according to an exemplary embodiment of the disclosed subject matter.

FIGS. 3A-3G show aspects of electrical stimulation of tissues according to an exemplary embodiment of the disclosed subject matter.

FIGS. 4A-4F show aspects tissue fabrication according to an exemplary embodiment of the disclosed subject matter.

FIGS. 5A-5F show aspects of video acquisition, electrical stimulation, and stage positioning of tissues according to an exemplary embodiment of the disclosed subject matter.

FIGS. 6A-6E show aspects of characterization of tissues cultured on the bioreactor according to an exemplary embodiment of the disclosed subject matter.

FIGS. 7A-7E show aspects of tissues cultured on the bioreactor according to an exemplary embodiment of the disclosed subject matter.

FIGS. 8A-8D show aspects of characterization of tissues cultured in the presence of subject sera on the bioreactor according to an exemplary embodiment of the disclosed subject matter.

FIG. 9 shows how myocarditis patients are collected and comprehensively characterized in the clinic according to an exemplary embodiment of the disclosed subject matter.

FIGS. 10A-10C show aspects of the platform development and cardiac tissue model characterization according to an exemplary embodiment of the disclosed subject matter.

FIGS. 11A-11B show aspects of identifying cardiac targets (according to an exemplary embodiment of the disclosed subject matter.

FIGS. 12A-12C show how engineered cardiac tissue performance correlates with patient diagnosis in adult and fetal myocarditis models according to an exemplary embodiment of the disclosed subject matter.

FIGS. 13A-13C show how combining calcium handling and force generation scores improves in vitro patient diagnosis according to an exemplary embodiment of the disclosed subject matter.

FIGS. 14A-14C shows that tissue stress leads to more pronounced differences between myocarditis patients and controls according to an exemplary embodiment of the disclosed subject matter.

FIGS. 15A and 15B show aspects of bioreactor fabrication according to an exemplary embodiment of the disclosed subject matter.

FIGS. 16A-C show aspects of batch to batch variability according to an exemplary embodiment of the disclosed subject matter.

FIGS. 17A-B show aspects of staining of a D100 cardiac tissue according to an exemplary embodiment of the disclosed subject matter.

FIGS. 18A-C show aspects of characterization of cardiac tissue according to an exemplary embodiment of the disclosed subject matter.

FIGS. 19A-C show aspects of staining of a D100 cardiac tissue according to an exemplary embodiment of the disclosed subject matter.

FIG. 20 shows aspects of incubating autoantibodies from patients' sera with engineered cardiac tissues in a bioreactor according to an exemplary embodiment of the disclosed subject matter.

FIGS. 21A-C show aspects of clinical data for a cohort of SLE patients according to an exemplary embodiment of the disclosed subject matter.

FIGS. 22A-K show aspects of engineering, maturation, and functional evaluation of human cardiac tissues according to an exemplary embodiment of the disclosed subject matter.

FIGS. 23A-J show aspects of determining associations between cardiac stress, SLE-myocarditis autoantibody binding levels, and the severity of myocardial inflammation according to an exemplary embodiment of the disclosed subject matter.

FIGS. 24A-I show aspects of determining whether specific autoantibody profiles can impact cardiac function independently from overall levels of autoantibody binding to tissues according to an exemplary embodiment of the disclosed subject matter.

FIGS. 25A-E show aspects of determining correlation of specific autoantibodies with in vitro tissue function according to an exemplary embodiment of the disclosed subject matter. LC-MS validation

FIGS. 26A-B show aspects of according to an exemplary embodiment of the disclosed subject matter.

FIG. 27 shows aspects of functional metrics calculated from contractility and calcium traces according to an exemplary embodiment of the disclosed subject matter.

FIG. 28 shows aspects of additional functional metrics compared for RPMI and MM media according to an exemplary embodiment of the disclosed subject matter.

### DETAILED DESCRIPTION

Engineered cardiac tissues derived from human induced pluripotent stem cells (iPSCs) are increasingly used for drug discovery, safety pharmacology, and as developmental and disease models for basic research. While there are numerous platforms to engineer cardiac tissues, they are often proprietary, require expensive and non-conventional equipment, and utilize parallel advanced video processing algorithms. As a result, only specialized academic labs have been able to harness this technology. In addition, methodologies and tissue features have been challenging to reproduce between different groups and across different models.

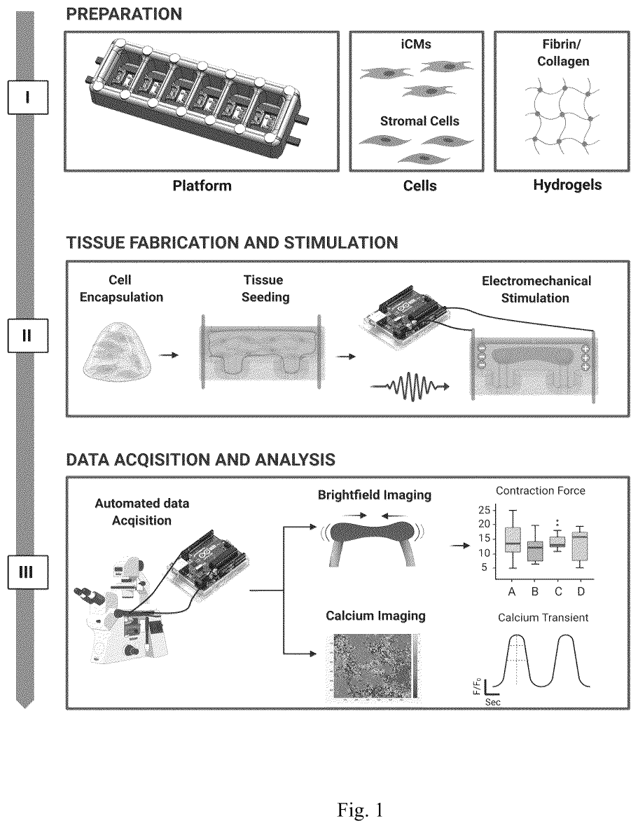

We have developed a platform for the culture of human engineered cardiac tissues composed of induced pluripotent stem cell (iPSC)-derived cardiomyocytes and cardiac fibroblasts. The platform fits within the footprint of a standard microscope slide, and comprises a plurality of culture wells arranged linearly between two carbon electrodes that are exposed within each well. Two horizontal parallel flexible pillars extend from each well so that an engineered tissue (composed of the above described cells within a 15 microliter fibrin hydrogel) can be suspended between each pair of pillars. Horizontal pillars allow us to measure pillar displacement along two dimensions, useful for accurate contraction, relaxation and work parameters. The platform allows for the fabrication, culture, and analysis in-situ in the same well without any further manipulation. Aspects of the platform are shown in FIG. 1, including platform preparation, tissue fabrication and stimulation and automated data acquisition.

Described herein are (i) reactor fabrication, (ii) tissue generation, (iii) electrical stimulation, (iv) a custom automated real-time data acquisition and (v) advanced video analyses of culturing engineered cardiomyocytes in the system described herein. We validate these methodologies and demonstrate the versatility of the platform by supporting the fabrication of tissues in different hydrogel materials, and with different cardiomyocytes derived from different iPSC lines and stromal cell types. We also validate the use of electrical stimulation for long term culture as well as a flexible automated force and calcium data analysis suite compatible with bright field and fluorescent imaging. Lastly, we showcase the compatibility of milliPillar with advanced electromechanical stimulation techniques to enhance cardiac tissue function. We expect that this platform for tissue generation and assessment will provide the scientific community with a valuable and accessible tool for generating customizable human engineered cardiac tissues and accelerate cardiac research and pharmacological studies.

Similarly, readouts of tissue functionality are an important point of consideration for studies using engineered cardiac tissues as well. Readouts from cardiac tissue models must meet the requirements of being quantifiable, reproducible, and relevant to the biological question at hand. With such a diversity of models that exist, it is imperative to be able to compare tissue functionality across different models and research projects; therefore, the use of absolute values is critical. Quantitative metrics extracted from calcium and brightfield images provide absolute values for calcium handling and force generation, metrics that are useful for both pharmacological screenings and studying cardiac diseases. Together with the system described herein, we have developed new algorithms to get quantitative metrics that can be compared across labs.

### Platform Fabrication and Mechanical Characterization

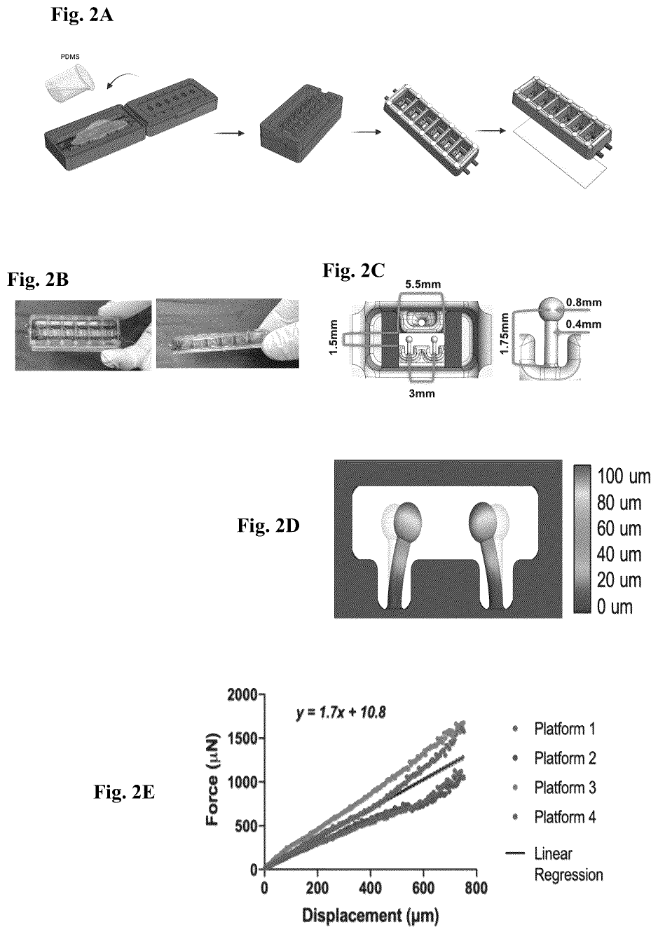

The bioreactor disclosed herein is fabricated from Polydimethylsiloxane (PDMS), due to its low cost, ease of manipulation, biocompatibility and gas exchange properties. PDMS also allows for easily covalent bonding to a glass slide to enable real-time brightfield and calcium imaging of the cardiac tissue in real-time in-situ. The bioreactor is designed to accommodate six tissues, cultured in separate wells to enable controlled culture conditions for each individual tissue (FIGS. 2A and 2B). The reactor also known as “milliPillar” was rationally designed at the mm-scale to decrease the number of input cells of current bioreactors, while at the same time keeping the reactor user friendly as mm-scale tissues are easier to manipulate in standard laboratory conditions compared to high throughput devices. The full reactor dimensions were designed to fit 4 reactors in one standard 4-well plate, enabling 24 tissues to be manipulated and analyzed at a time. Notably, the spacing between the 24 tissues is compatible with a 96-well configuration to enable assays designed for real-time assays in microplate readers. The dimensions of each culture well (for example, 13×7×6 mm) were designed to accommodate small amounts of culture media (400 ul) in order to maximize the charge injected per unit volume and to enable studies which may require precious media supplements (e.g. patient serum, exosomes, growth factors and cytokines.) (FIG. 2C).

Flexible anchors (e.g pillars, posts, wires) provide passive tension along the tissues for continuous mechanical strain, and consequently auxotonic tissue contraction. Here, we designed, mm-sized pillars (pillar head diameter; 0.8 mm and stem length; 1.75 mm) that were placed in a horizontal configuration at the bottom of each well to improve stretching and attachment of tissues around the pillar heads and improve pillar deflection visualization to accurately calculate tissue force generation (FIG. 2C). We modeled this movement to show the linear deflection of the pillars, where we calculate the deflection based on the center of the pillar yellow region=80 um, FIG. 2D). To our knowledge, this is the first study reporting the use of horizontal pillars, which allow for complete observation of the pillar shaft and tissue movement.

The mechanical properties of the PDMS pillars are sensitive to curing temperature and time, ratios of base and curing agent, and can even change with time at room temperature. Using simple brightfield imaging, we were able to track the pillar movement, as well as detect the pillar deflection at rest, due to the horizontal orientation of the pillars. The derived coefficient from the linear regression of the force as a function of displacement allows for direct calculations of active forces and passive tensions based on the displacement of the pillars by cardiac tissues during contraction or at rest (FIG. 2E). Our results demonstrated that among four different batches, the pillars reproducibly exhibit linear elastic behavior over the testing range (0-750 um, FIG. 2F). The pillar exhibits no elastic hysteresis suggesting both tissue contraction and relaxation can be evaluated with the same coefficient.

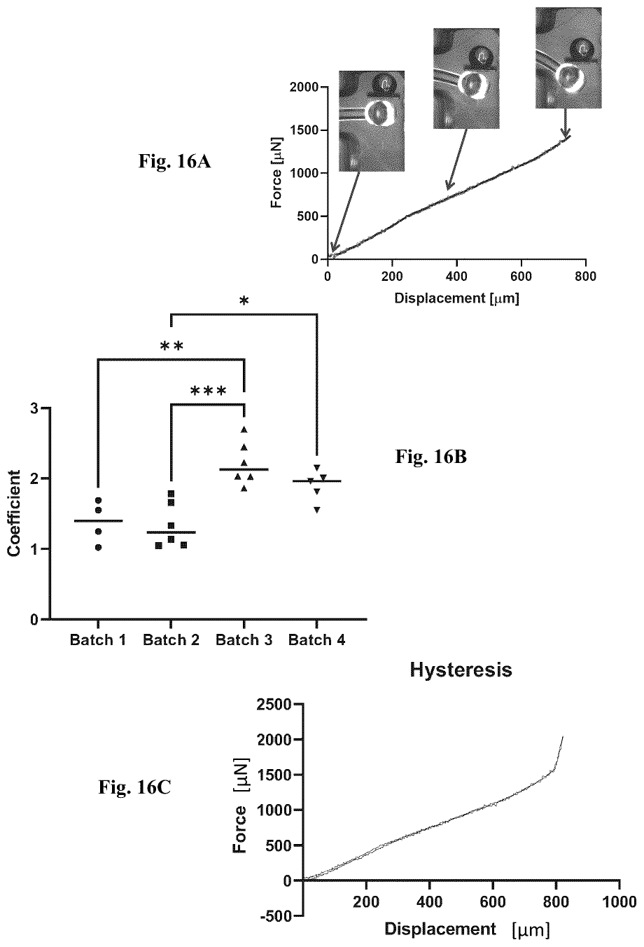

As anticipated, there is batch to batch variation (FIGS. 16A-C). Our results indicate the importance of keeping all PDMS fabrication parameters consistent, to minimize variability. Ideally, a large batch of reactors should be made at the same time and used soon after fabrication to minimize variability. Users should also note that PDMS is known to absorb hydrophobic compounds and drugs and therefore can hamper its use in certain studies. Future studies involving the investigation of other biocompatible materials would be invaluable.

The system described herein uses a customized electrical stimulation apparatus to facilitate a controlled charge injection. Many groups including our own have adapted the in vivo phenomena of cardiac muscle contraction driven by electrical coupled pacemaker cells and translated it in vitro. It was demonstrated that electrical stimulation improves cell-cell connectivity, alignment and overall tissue performance. Our group has also modeled field stimulation for the use of cardiac tissue stimulation and that carbon is a superior material choice for electrical stimulation. We built upon this theory to incorporate a double bi-layer capacitor model. We designed the milliPillar carbon rods to create a double bi-layer capacitor and we were able to calculate the exact amount of charge injected into our media. Pure carbon rods were placed 5 mm apart and are an improvement from rods used in the past, due to the elimination of resin used in the carbon rods of previous studies, which are more porous (increasing surface area) and more conductive; both of these properties enhance the amount of charge injected into the media, per pulse.

Normally, electrical stimulation of engineered cardiac tissues requires expensive and specialized equipment, not commonly available in most research laboratories. To address this barrier, we have developed an inexpensive, user-friendly, and easily customizable device built from commercially available components and an open source circuit board.

The use of our custom stimulator allows for precisely controlled and recorded electrical stimulation at any point during tissue studies. Customizable stimulation regimens can be used (i) during culture to promote the maturation of tissues, as previously described, and (ii) for real-time non-destructive functional characterization of the tissues at any point during culture without the need for additional equipment or the transfer of tissues to external devices. Stimulating during functional assessment can standardize measurements between tissues with different beating frequencies and provide insight into important cardiac tissue characteristics, such as the force frequency response and frequency dependent acceleration of relaxation, that require dynamic beating frequencies.

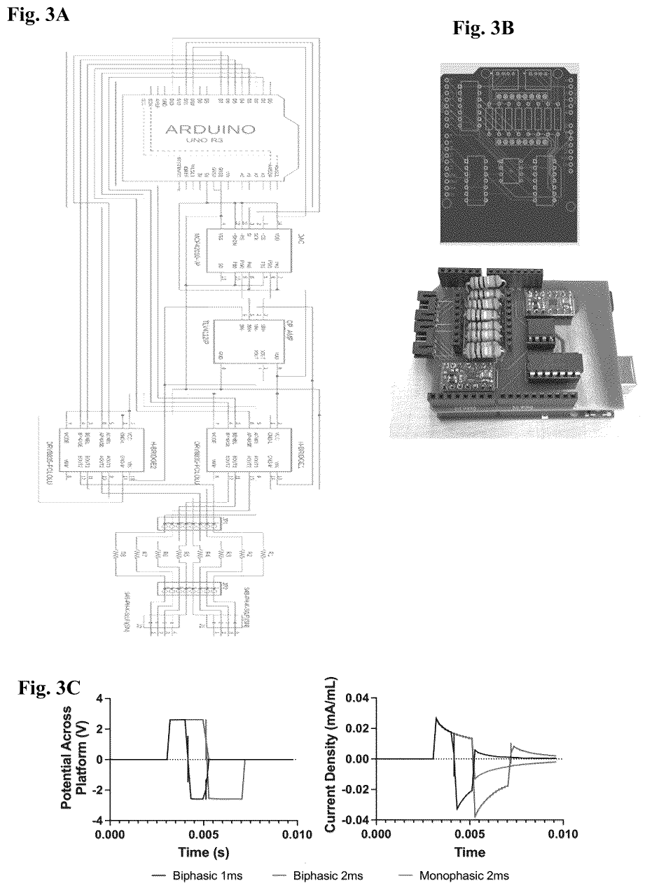

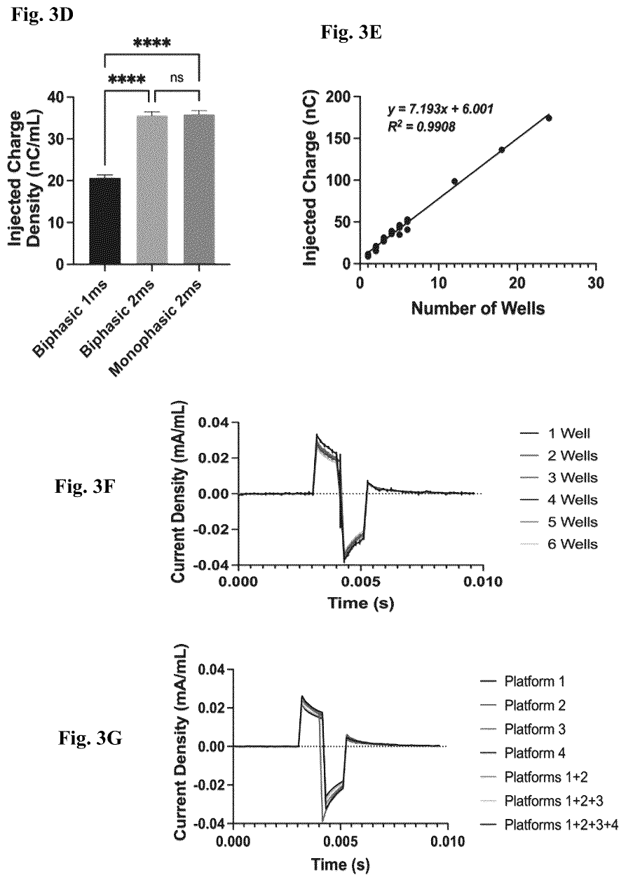

We have extensively validated the system to ensure that it can provide sufficient charge injection for the milliPillar reactor as determined by previous studies. Importantly, we have incorporated a current and voltage measurement system to provide quality control over the stimulation regimen for the duration of tissue stimulation (FIG. 3B). We have expanded our use of an Arduino Uno to reproducibly inject a safe amount of charge that is compatible with cardiac tissue stimulation (FIG. 3A). We have expanded its capabilities to create either a biphasic or a monophasic wave. Here we show the voltage and current of one waveform of a 2.5V (generating a 5V/cm electric field) output at 2 Hz measured across the reactor (FIG. 3C). The current across the reactor is dependent on how many wells are in use in each reactor. We found that per well, there is additional current running through the system, due to the increased pull from additional electrolyte/carbon surface area. This equates to an average of ˜.03 mA/mL current per pulse (FIG. 3D). When using only one well of the reactor, we found that the current per pulse was elevated (˜.04 mA/mL). When measuring multiple reactors, we see that there is negligible batch to batch variation and the charge injected per well is ˜.025-.03 mA/mL (FIG. 3E). Our results indicate that each channel on our Arduino device can output a controlled ˜.012 mA current/400 uL well for 4 reactors maintaining an electric field of 5V/cm. Previous studies have shown the injection of charge into a large reservoir of media containing many tissues, making it difficult to control the amount of charge to which each tissue is exposed. To our knowledge, we are the first group to characterize the amount of charge injected into an isolated cardiac tissue well. This allows us to reproducibly expose each cardiac tissue to the same amount of electrical stimulation and allows for the detection of more nuanced differences in research projects. Importantly, as electrical stimulation generates oxidative stress within the cell, and at extremes can create faradaic currents, it is important to control the charge injected to stimulate the cardiac constructs in a sustainable manner that preserves the health of the tissue.

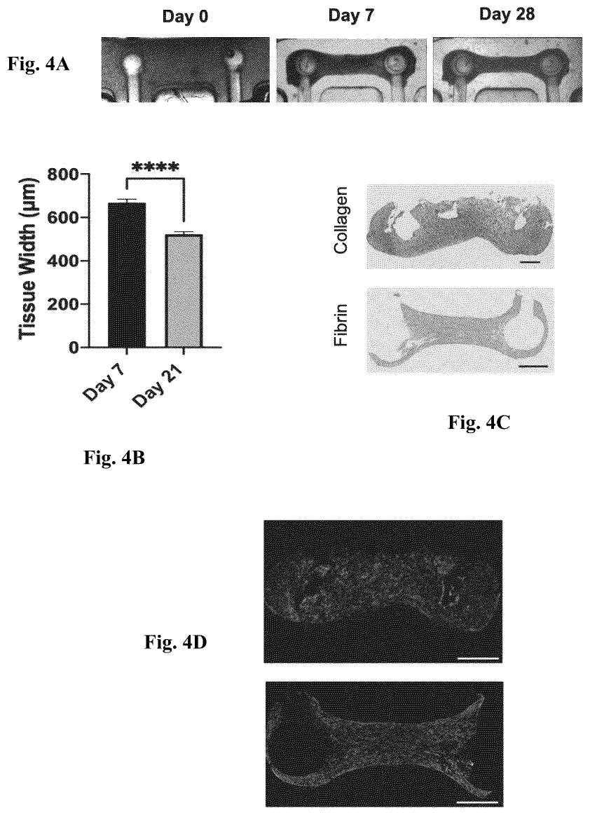

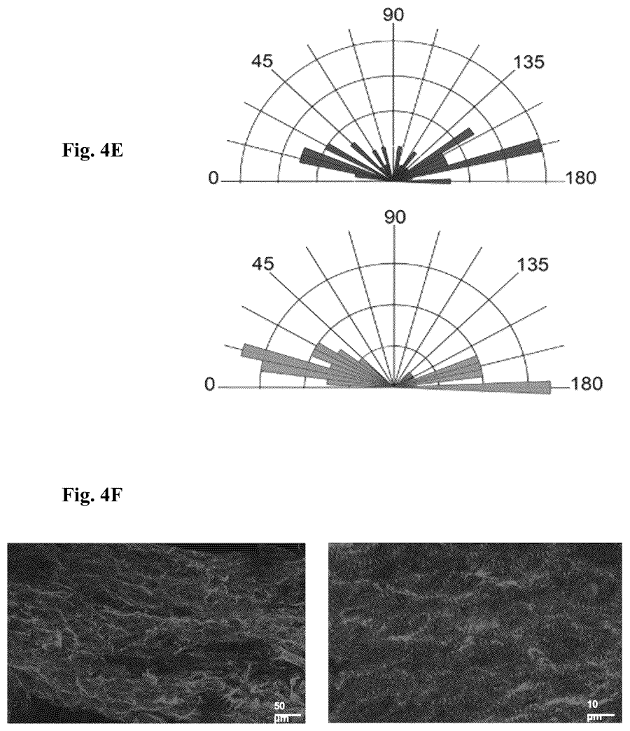

Engineered cardiac tissues were fabricated by mixing cardiomyocytes and cardiac fibroblasts within a hydrogel and cast into molds. The area in which the cell-laden hydrogel is cast was designed to minimize tissue size, requiring only 10-15 μL in volume and approximately 500,000 cells. During the first 7 days, the cells extensively remodeled the hydrogel and formed compact tissues surrounding the pillar heads (FIG. 4A). In the following 3 weeks in which the tissues were electrically stimulated, further compaction was observed (FIG. 4A), with significant difference in tissue width before and after stimulation (.668 mm, .522 mm, p<.00001, FIG. 4B).

It has previously been demonstrated by our group and others that different hydrogel materials such as collagen, fibrin, and decellularized ECM support functional cardiac tissue assembly. Therefore, we sought to evaluate the versatility of millPillar by generating cardiac tissues in both collagen and fibrin hydrogels. Our results demonstrated that in both collagen and fibrin hydrogels, the cells remodeled the hydrogel and formed compacted tissues with aligned cardiomyocytes as observed by histological staining and immunofluorescence imaging (FIGS. 4C and 4D). Quantification of cellular alignment demonstrated that in the fibrin hydrogels more alignment, perpendicular to the pillar axis, was observed than in the collagen hydrogels (FIG. 4E).

The milliPillar reactor gives the user the option to use electrical stimulation as a method to promote maturation of cardiac tissues. To evaluate the effect of sustained biphasic electrical stimulation provided by the custom stimulator on milliPillar tissues, we electrically stimulated tissues for 3 weeks with an intensity training regimen as previously reported. Immunofluorescence staining for alpha-actinin, demonstrated the formation of pronounced striations and cell alignment indicative of an improved contractile apparatus (FIG. 4F).

The choice of iPS-CMs lines, type of stromal cells, and ratio of cardiomyocytes to stromal cells differs between protocols and different research groups and according to the question in hand. Our goal here was to develop a technology that is customizable and lends the researcher the highest amount of flexibility. Therefore, we sought to investigate its ability to form tissues with various cell types and ratios. Our results demonstrated that functional tissue assembly was achieved using different iPS-CMs lines and different types of stromal cells (iCFs and dermal fibroblasts. Determining the superior choice of cell source exceeds the scope of this study and is left for the user to determine based on the constraints of their research project. Finally to demonstrate the potential of the platform to be used for long-term studies we cultured milliPillar tissues for 100 days and Immunofluorescence images revealed pronounced actinin striation.

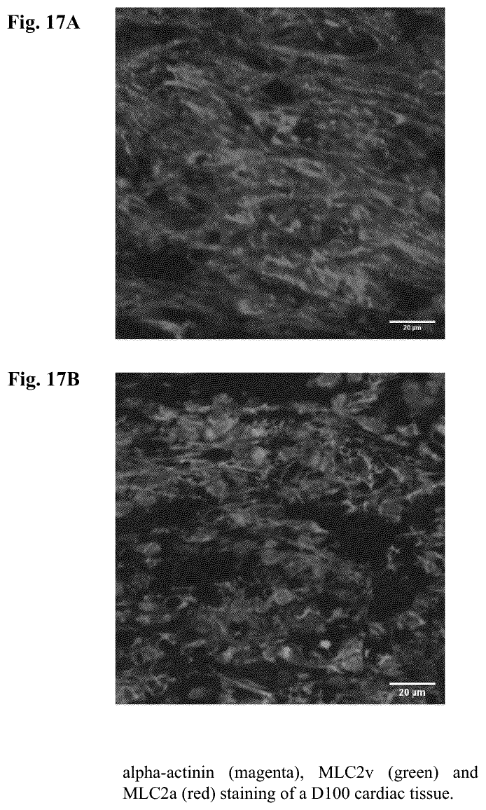

We also explored the ventricular phenotype of the milliPillar tissues. MLC2v staining indicates that our tissues facilitate ventricular cardiomyocyte differentiation, which increases over time (FIGS. 17A-B). While further validation is required, there is ample reason to believe that atrial tissues can be generated by adapting this methodology with atrial cardiomyocyte differentiation protocols.

The disclosed system uses custom software and hardware for the functional analysis of cardiac tissues. Monitoring cardiac tissue functionality, non-invasively, over extended periods of time is important to evaluate its response to various pharmaceutical compounds, environmental signals, and also in understanding their development. Optical imaging provides an ideal solution for this need since it is non-destructive and could be translated into absolute values of tissue functionality. In addition, automated data acquisition is ideal to improve study throughput and reduce user error.

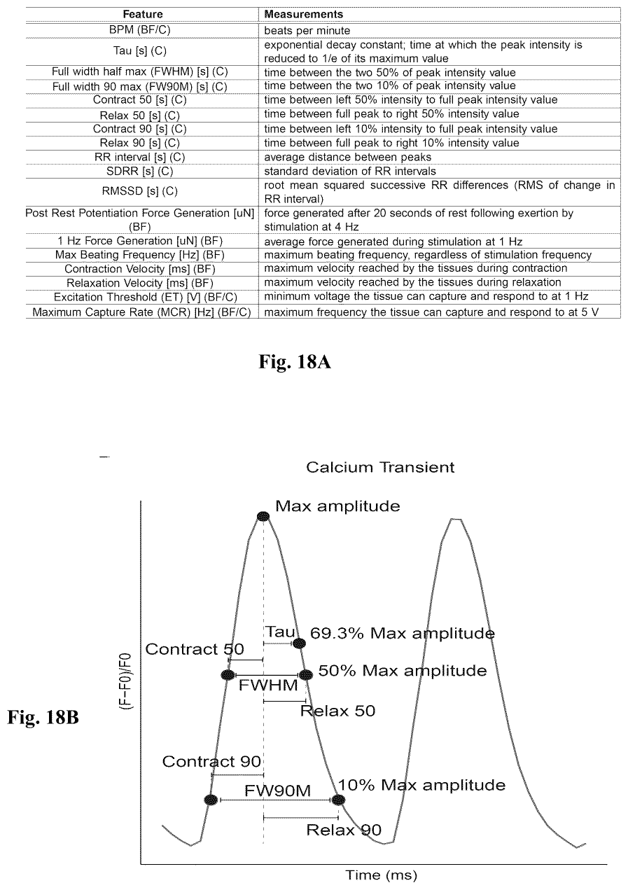

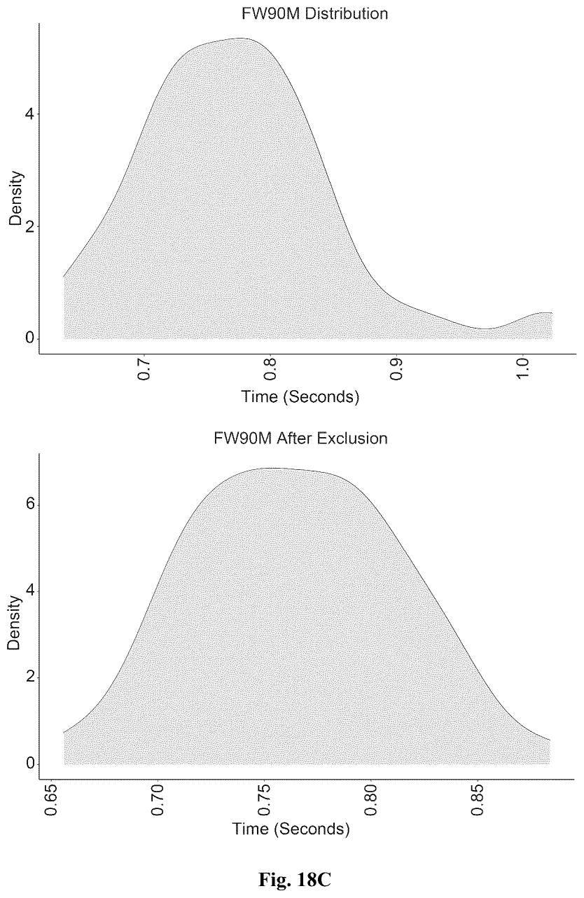

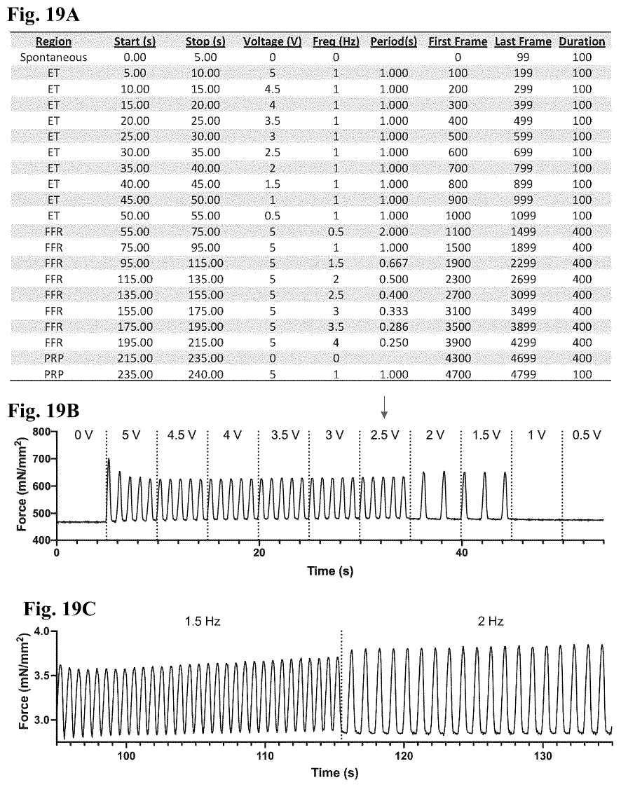

To address this need, milliPillar is complemented by a custom software suite that allows for automated controlled measurements of force generation, excitation threshold, maximum capture rate and analysis of individual calcium transients. Separately, we have used established methodologies for calcium and force measurements to implement custom software that analyzes the milliPillar cardiac tissue functionality. A summary of the measurements and their meanings are provided (FIG. 18).

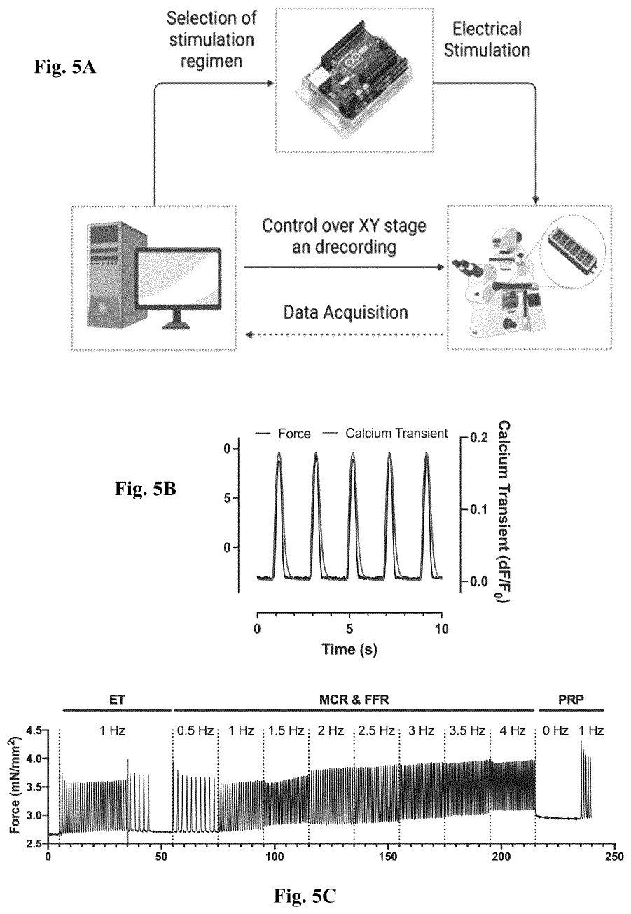

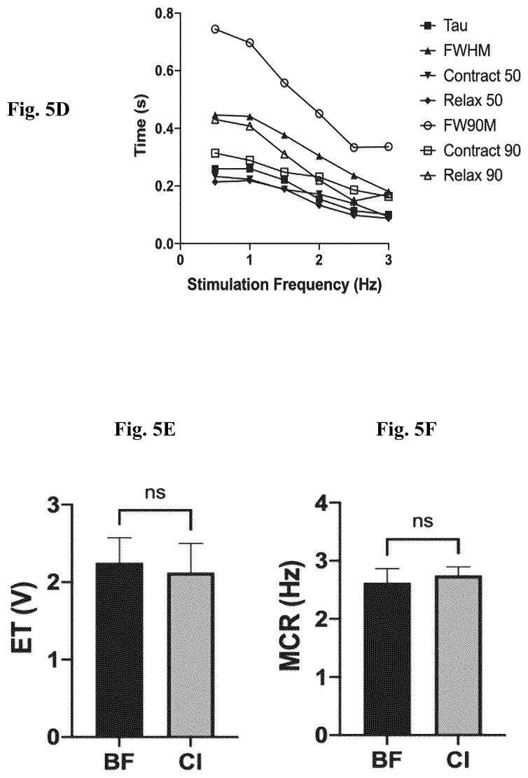

We have equipped a standard microscope with a custom built stimulator and custom software to control and synchronize video acquisition, electrical stimulation, and stage positioning (FIG. 5A). This allows for assessment of up to 24 tissues at a time. Calcium transients were measured with an iPS-GCaMP cell line, genetically encoded with a calcium indicator to allow real-time readouts. In our studies, we used the calcium and force transients recorded under 1 Hz electrical stimulation to assess cardiac function. Representative calcium and force transients are shown at 1 Hz (FIG. 5B).

The milliPillar stimulator stimulates the tissue during bright field imaging to determine the ET, MCR, FFR, and PRP in a single automated recording (FIG. 5C). During this recording, the stimulation regimen begins without any stimulation to record the spontaneous beating activity and then begins 1 Hz stimulation to measure the ET. The stimulation voltage begins at 5V and then drops every 5 seconds so that the analysis program can determine the voltage at which the tissue stops responding to stimulation. For the MCR and FFR, the voltage is fixed at 5V and then the stimulation frequency increases by 0.5 Hz every 20 seconds, allowing the program to determine the frequency at which the tissue stops contracting with every stimulus (MCR) and measure the deflection of the pillars to calculate the force generation as the frequency increases (FFR). To measure the PRP, the stimulation pauses for 20 seconds after the MCR/FFR frequency ramp and then resumes at 1 Hz. The force generated by the first beat upon the resumption of stimulation is the PRP.

As the stimulation frequency increases, the maximum force generation of each contraction also increases, demonstrating a positive FFR, a physiological feature of mature cardiac tissue (FIG. 5C). It should also be noted that the baseline passive tension that the tissue reaches during relaxation between beats also increases with increasing stimulation frequencies, demonstrating that the tissue is unable to fully relax to its original length between beats at high frequencies. This observation makes sense given the likely inability of the cardiac contractile mechanism to fully recycle calcium and relax completely during the artificially shortened period between beats prompted by supraphysiological pacing.

In addition to positive FFR, cardiac tissues exhibit a frequency dependent acceleration of relaxation (FDAR) and can be recorded at different frequencies of stimulation (FIG. 5D). The versatility of the system to stimulate across a wide range of frequencies is important given the frequency dependence of many phenotypes and drug responses. Such features may only become apparent during stimulation at frequencies that recapitulate either bradycardia or tachycardia.

By directly measuring the deflection of pillars calibrated with known bending coefficients, the milliPillar system enables the calculation of absolute values of force generation, active force, and passive tension, rather than the relative approximations generated by some systems. This facilitates the reporting of uN/mm², which is becoming a requirement asked for by various consortia and regulatory agencies. It should be noted, however, that the level of force generation by tissues within the milliPillar platform and reported by the analysis suite is not necessarily the maximum force generation achievable by the tissues, due to the Frank-Starling positive relationship between cardiomyocyte length and force generation. The tissues are not necessarily stretched to their optimal relaxed length to maximize force generation within the milliPillar bioreactor since the passive tension and stretch, which correlate to afterload in vivo, are not adjustable by the user. This is a trade-off for the ease of real-time measurements enabled by the bioreactor that can be conducted over time throughout the course of a study. At the study endpoint, tissues can be removed from the bioreactor and subjected to standard force recordings within a traditional organ bath system.

The analysis suite can also be set to calculate the ET and MCR with brightfield or fluorescent imaging. We found that brightfield and calcium imaging showed no difference in the method used to assess ET and MCR (FIG. 5E). Although ET and MCR using calcium transients are easier to analyze, due to the high signal to noise and ease of computation processing, it is important to note that blue light is toxic, and overexposure may lead to confounding effects. Calcium transient features (tau, FWHM, etc.) can only be calculated with fluorescent imaging, however, so care must be taken to not overexpose tissues with toxic blue light.

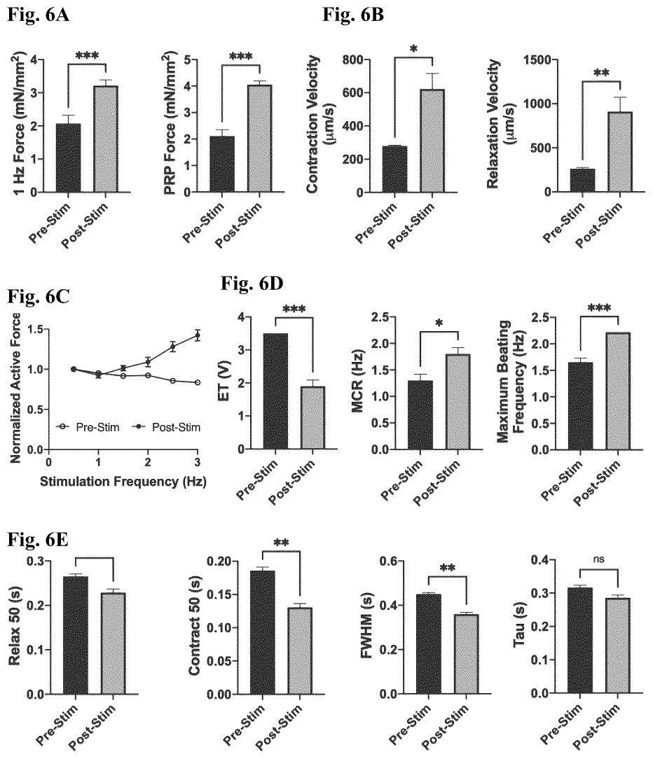

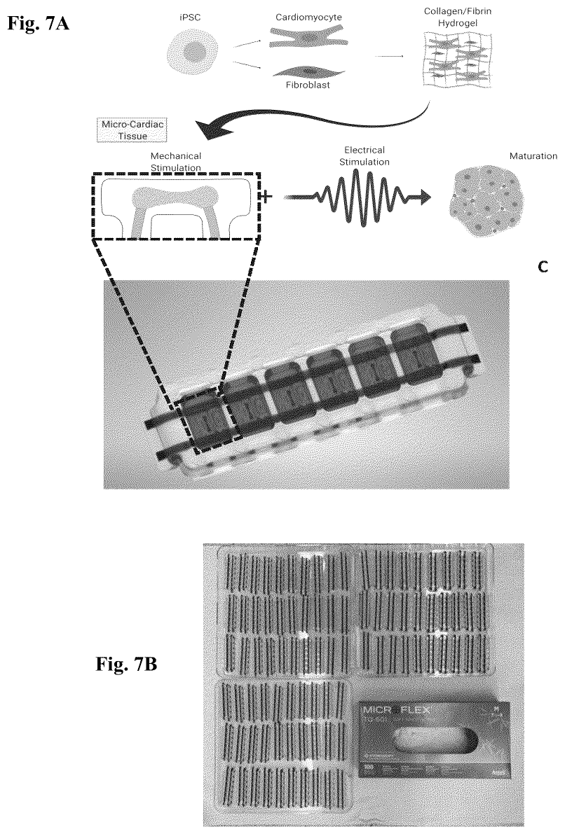

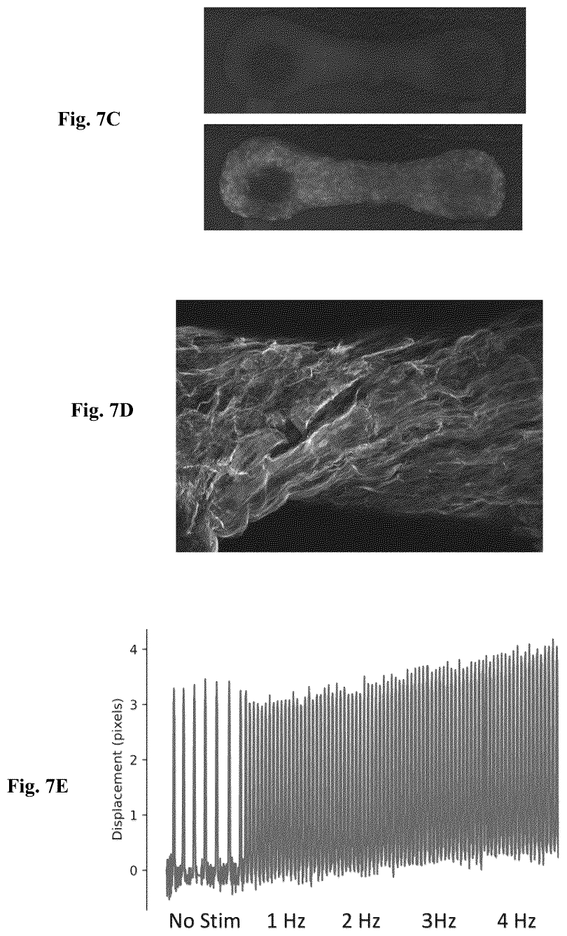

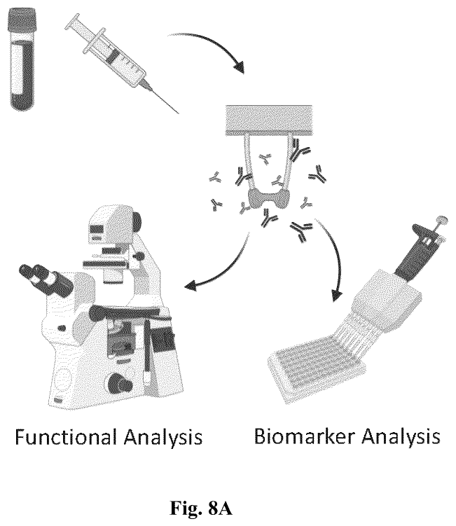

Electromechanical stimulation enabled by milliPillar enhances cardiac tissue function. After the fabrication of tissues, milliPillar provides the capability to enhance tissue function with its customized stimulation arduino. Ramp stimulation has been shown to enhance tissue functionality¹², therefore we used this methodology and tracked the milliPillar tissues over time. As expected, milliPillar tissues showed enhanced force generation, with both 1 Hz force generation (247 uN vs 396 uN, p=<.001) and PRP (425 uN vs 548 uN, p value=<.01) increasing between baseline and post stimulation (FIG. 6A). Accordingly, the maximum contraction velocity (280 um/s vs 622 um/s, p<0.05) and relaxation velocity (263 um/s vs 911 um/s, p<0.05) also increase after ramp stimulation, demonstrating more mature and physiologically functional tissues. Mature ventricular cells exhibit shorter calcium transients as their sarcoplasmic reticulum is organized and saturated with Ryanodine Receptors and SERCA2A calcium channels. A more functional sarcoplasmic reticulum is able to more rapidly release and reuptake calcium during every beat. Consistent with this, calcium handling was found to be enhanced as shown by decreases in Contraction 50 (131 ms vs 186 ms, p=.00046), Contraction 90 (184 ms vs 248 ms, p=.00028), FWHM (350 ms vs 450 ms, p=.00023), FW90M (678 ms vs 744 ms, p value=.0033), Tau (317 ms vs 286 ms, p=.032), and Relax 50 (229 ms vs 265 ms, p=.015) (FIG. 6B).

The excitation threshold (ET), which is used to assess the electrical excitability and sensitivity of the tissue to electrical stimulation, was markedly decreased as you would expect from a more mature cardiac tissue (3.5 V vs 1.9 V, p<0.001) (FIG. 6C). A cardiac tissue that can respond to increasing frequencies is indicative of a more functional sarcoplasmic reticulum, sarcoplasm and sarcomere. As with other metrics we found that electrically stimulated tissues exhibited higher maximum capture rates (1.3 Hz vs 1.8 Hz, p<0.05) and higher maximum beat frequencies (1.7 Hz vs 2.2 Hz, p<0.005) (FIG. 6D). A positive force frequency response, an important feature of mature cardiac muscle in vivo, is observed after stimulation, but not before, indicating functional maturation over the course of the stimulation regimen (FIG. 6E).

We envision that milliPillar and/or LC-MS would be able to be used in the clinical setting to diagnose patients with subclinical myocarditis, help improve myocarditis-heart disease risk stratification and management for precision medicine, to ultimately improve cardiovascular outcomes in myocarditis. In addition, this technology could open a new avenue for identifying autoimmune mediated mechanisms of heart disease.

In a preferred embodiment, the dislcosed system can be used to diagnose subclinical myocarditis in patients with systemic lupus erythematosus (SLE) and Rheumatoid Arthritis (RA).

The 1.5 million Americans who live with systemic lupus erythematosus (lupus) are three times more likely than the general public to develop heart failure, often as a result of myocarditis. Diagnosis and treatment of early stage myocarditis (ES-myocarditis) in lupus prevents the onset of heart failure; however, current methods used to diagnose ES-myocarditis in lupus are inaccurate, unreliable, or inaccessible due to cost. There is an unmet clinical need to detect ES-myocarditis in lupus. To address this clinical need, we have developed a highly sensitive and specific diagnostic tool that can identify ES-myocarditis in lupus, which would otherwise go undetected. Using serum, the method can report results in less than 24 hours, aiding in patient-specific risk stratification and facilitating etiology-based therapies. Diagnosis of ES-myocarditis will reduce heart failure incidence, thus reducing morbidity, mortality and healthcare costs by $9.5B annually.

Systemic lupus erythematosus (lupus) is a complex autoimmune disease that results in a variety of disease manifestations and tissue injury, often leading to end stage organ failure. Approximately 1.5 million Americans are currently living with a form of lupus. The standardized mortality ratio for lupus patients compared with the general population is 3.6, with rates as high as 19.2 in 16-24 year olds. Heart failure (HF) is a leading cause of this excess mortality, and in lupus patients it often develops from autoimmune-mediated damage of myocardium, a condition known as myocarditis. Given the heart's lack of regenerative capacity, once HF manifests clinically, little can be done to regain function. The cost of HF in lupus is estimated to be $50K+per patient, a total of $9.5B annual costs in the US.

Systemic lupus erythematosus (SLE) is a highly heterogeneous autoimmune disease characterized by a diverse repertoire of autoantibodies. Cardiovascular disease (CVD) is the leading cause of morbidity and mortality in these patients, a clinical finding that is only partially explained by traditional CVD risk factors. The SLE itself is an independent risk factor with direct autoreactivity targeting cardiac tissue leading to myocardial inflammation (i.e. myocarditis) being considered a culprit. The paradigm of autoantibody-mediated cardiac injury is supported by the association of anti-SSA/Ro antibodies with congenital heart block and myocarditis. Clinical manifestations of myocarditis in adult SLE patients are highly variable, spanning from asymptomatic presentation to heart failure. It is thought that specific autoantibody signatures may explain a significant fraction of the observed clinical and phenotypic variations. Therefore, elucidating the mechanisms of pathogenesis of autoantibodies involved in SLE-myocarditis could lead to the discovery of etiologic factors, improve patient risk stratification, and aid in the development of new therapies.

There is an emerging consensus that many lupus patients develop such myocarditis long before it presents with clinical symptoms and the onset of HF. Our group and others have utilized advanced imaging techniques to determine that myocarditis occurs in approximately 25% of adults with lupus with no clear clinical cardiac manifestation. According to a report published by the European Society of Cardiology in 2017, the lack of cardiological diagnostic work-up and tools to diagnose lupus patients with early-stage myocarditis (ES-myocarditis) leads to patient under-treatment, and poor patient prognosis.

Despite the increasing evidence that lupus patients would benefit from the detection of ES-myocarditis, such diagnoses rarely occur in practice. Endomyocardial biopsies and FDG PET-CT scans are currently used clinically to diagnose autoimmune-mediated myocarditis, though neither is used as a screening tool for asymptomatic patients, and the invasiveness, lack of sensisitivity, and cost markedly limit their routine use in clinical practice. Endomyocardial biopsies are considered the gold standard, however they are not routinely performed due to the invasiveness of the procedure and are highly inaccurate as we recently reported. Echocardiography and electrocardiography are performed only after the patient presents with symptoms, and they lack sufficient sensitivity and specificity for screening. Sophisticated imaging techniques such as cardiac MRI and FDG-PET are more accurate, however they involve radiation and are not covered by insurance companies due to exorbitant costs. However, endomyocardial biopsies and FDG PET-CT will serve as clinical references for assessing the accuracy of our platform. Numerous studies have tried to identify biomarkers to non-invasively detect myocarditis in at-risk patients; however, the results are inconclusive.

The problem is that 15% of lupus patients develop myocarditis leading to heart failure, yet lack of reliable tools to detect ES-myocarditis leads to improper treatment of these patients and therefore increased morbidity and mortality rates. Therefore, there is a critical clinical need to diagnose ES-myocarditis in lupus patients to allow patient-specific risk stratification and treatment to reduce HF hospitalization rates and overall economic burden on the healthcare system. The ability to detect ES-myocarditis would influence the patient's treatment plan, ultimately avoiding the devastating effects and costs of HF.

A diagnostic tool that is (i) more effective than an EKG and echocardiograms at detecting ES-myocarditis, (ii) and is priced at less than $1200 would be desirable for early detection of myocarditis.

The capability of current models to recapitulate patient-specific interactions of autoantibodies with the adult human heart remains limited. Translating data from animal models to humans is challenging, due to species-specific differences in cardiac and immune physiology. The techniques for engineering human heart muscle from human induced pluripotent stem cells (hiPSCs) and their subsequent maturation by electromechanical stimulation to recapitulate many aspects of the metabolism, ultrastructure, and physiology of adult myocardium described herein are useful in modeling the role and mechanisms of action of autoantibodies in the pathogenesis of SLE myocarditis.

Cellular stress, often leading to apoptosis, is widely hypothesized to modulate autoimmunity in SLE, and previous studies have demonstrated that stressed cells are immunologically distinct from healthy ones. Plasma membrane rupture during apoptosis exposes intracellular antigens to the extracellular immune milieu. Additionally, the cellular stress and apoptosis have been shown to trigger the redistribution of intracellular antigens to the plasma membrane where they are immunologically accessible. Specifically, it has been reported for cardiomyocytes that oxidative stress alters gene expression, posttranslational modifications of proteins, and the cell surface distribution of antigens, rendering stressed cardiomyocytes immunologically distinct from those not stressed. Therefore, we hypothesized that myocardial stress could lead to differential autoantibody binding patterns to cardiac tissue and modulate autoantibody-mediated myocarditis. To test this hypothesis, we electrically paced the tissues at supraphysiological levels to induce oxidative stress and explored how autoantibodies affect tissue function during the periods of stress and recovery.

To address this clinical need, we have developed an in vitro diagnostic tool that uses a 100 μL serum sample to determine the presence of ES-myocarditis. Our device leverages advances in engineering and medicine to enable regular screening for ES-myocarditis. We differentiate from other technologies by (i) significantly improving accuracy when compared to gold-standard methodologies, (ii) providing a higher throughput and affordability when compared to imaging techniques and (iii) reducing the risks associated with radiation and heart biopsies. Backed by preliminary results, we believe AutoDetect can detect ES-myocarditis and revolutionize the care of lupus patients, drastically reducing the clinical and economic burden of HF in lupus.

Our solution is to generate a diagnostic tool consisting of engineered cardiac tissues that will be cultivated with patients' serum and analyzed in a high-throughput manner to identify the currently undetectable ES-myocarditis. Since our group's publication in Nature, reporting on engineered-human cardiac tissues capable of predicting patho(physiology) and drug responses, we have (i) scaled the technology to enable high-throughput screening, (ii) designed a prototype reactor as described herein, which is rapidly fabricated and drastically reduces costs, and (iii) enabled automated imaging for robust, reproducible, and low maintenance assessment. We demonstrated the capability of this device to generate functional human cardiac tissues with outputs that directly correlate to clinical benchmarks in both the healthy and diseased heart. Specifically, the tissues contract between flexible pillars, allowing imaging and analysis algorithms to calculate 10 functional metrics, including force generation, calcium dynamics, and biomarkers that directly correlate with the natural state of the human heart. These parameters greatly increase the accuracy and reliability of our diagnostic tool.

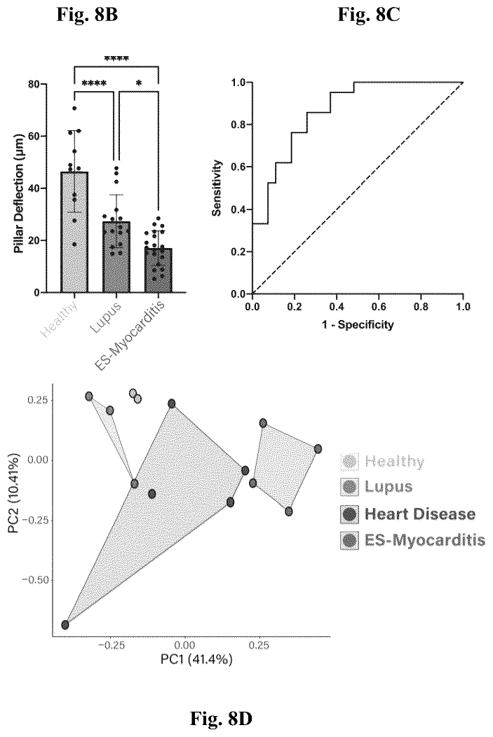

We screened 18 patients from Columbia University's Lupus Center and were able to differentiate between samples from lupus patients with and without ES-myocarditis, (FIGS. 8A,B) with 86% sensitivity and 74% specificity (FIG. 8C) compared to that of FDG-PET which is 74% and 97%, respectively.

To maximize the accuracy of the method, we used machine learning techniques to proportionally weigh the predictive contributions of each functional metric and of concentrations from a small panel of biomarkers. Preliminary results applying such methods to our data set have yielded clear clustering of each patient classification group (FIG. 8D).

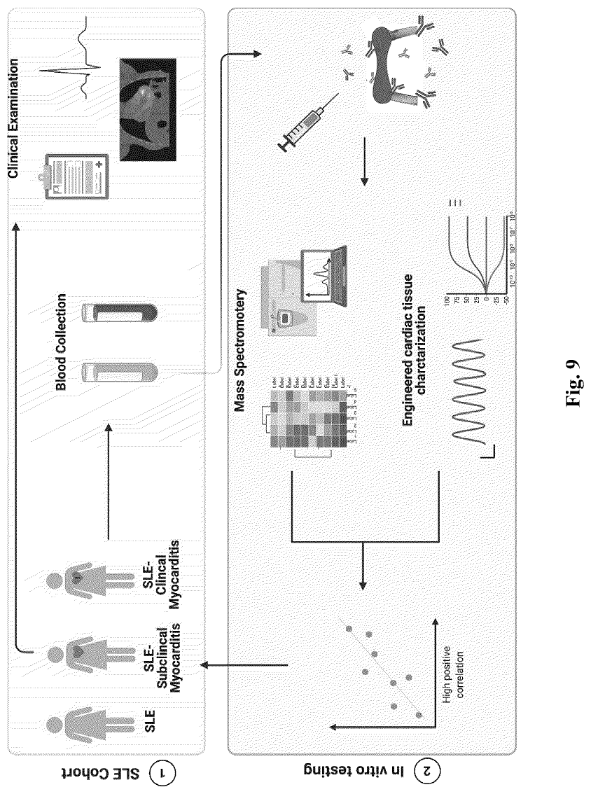

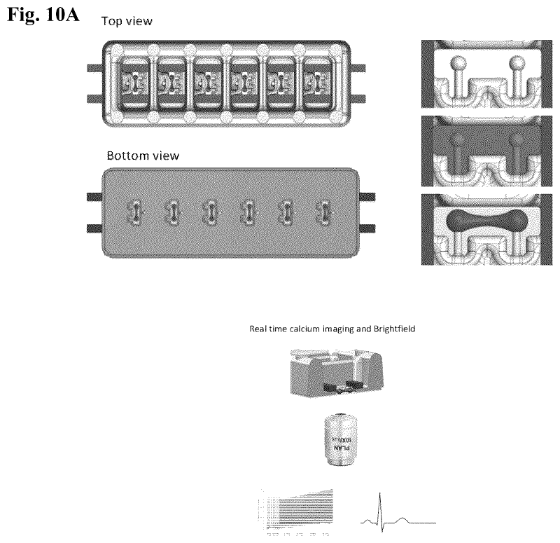

FIG. 9 shows how myocarditis patients are collected and comprehensively characterized in the clinic. (2) In vitro assays are used to investigate cardiac tissue functionality upon culture with patient serum in order to diagnose patients with myocarditis.

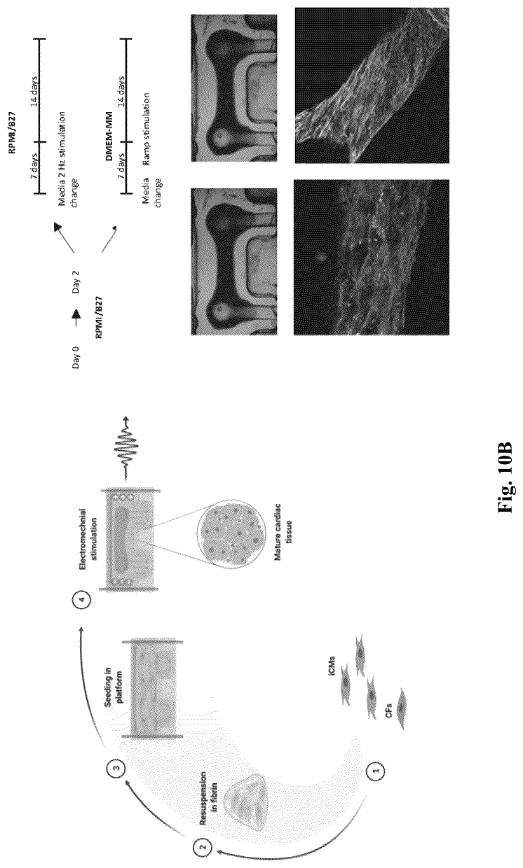

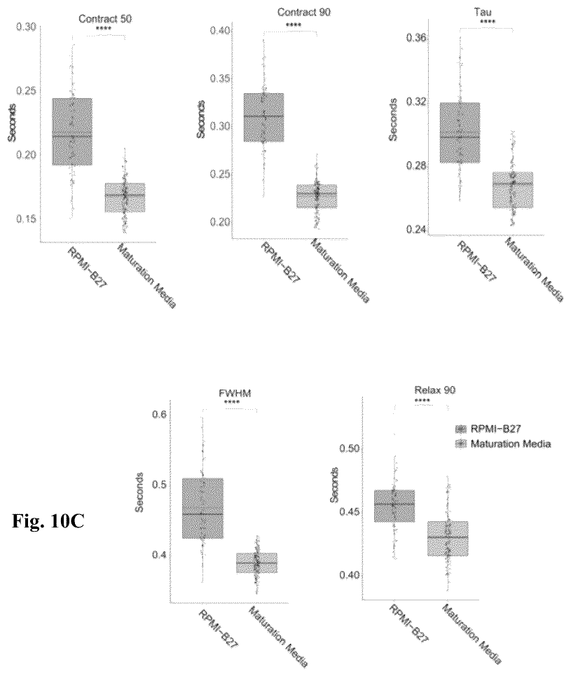

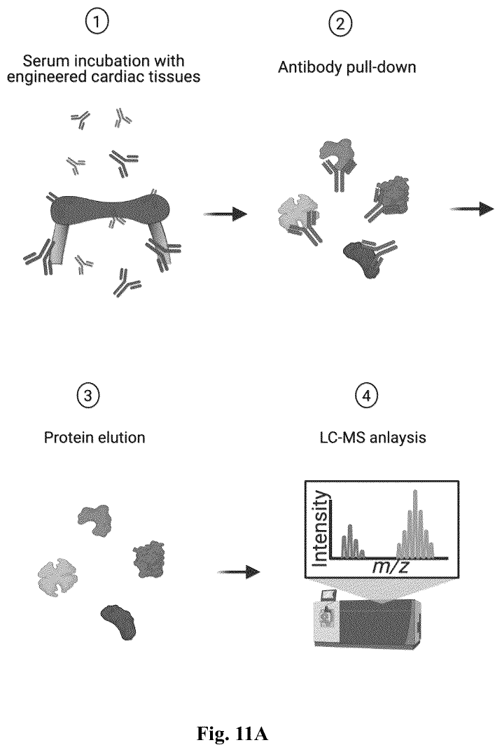

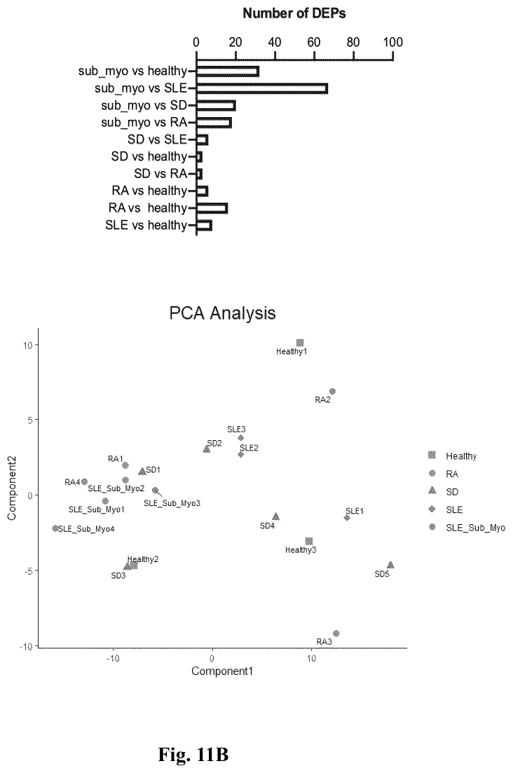

FIGS. 10A-B show aspects of the platform development and cardiac tissue model characterization (10A). The platform design is suited for medium, throughput studies and real-time imaging. (10B). Characterization of two different cardiac tissue models that will be used to diagnose patients

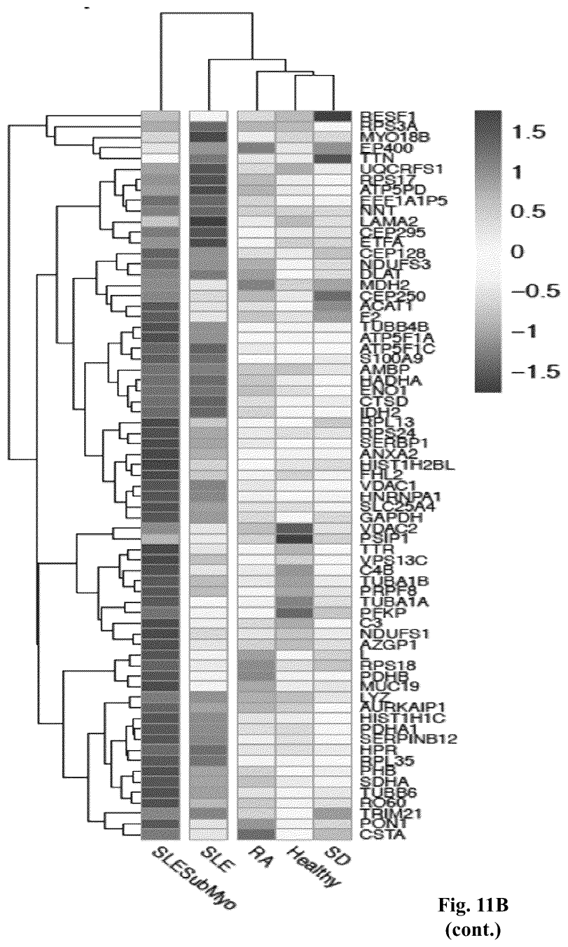

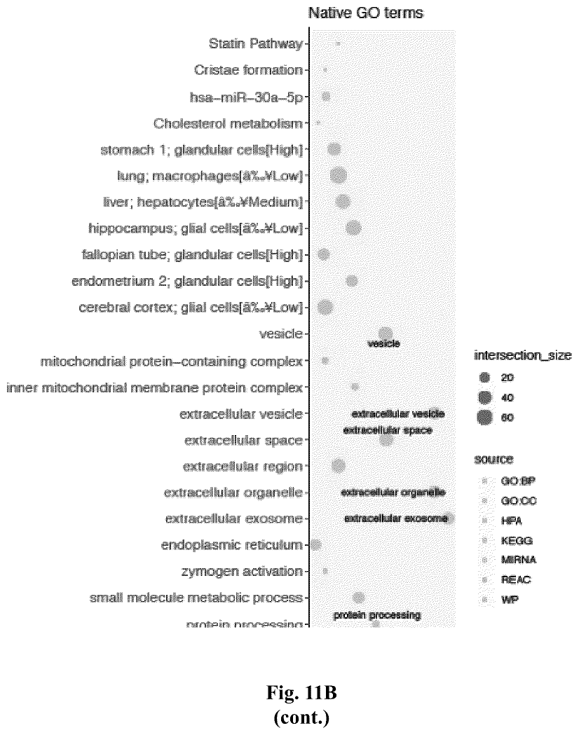

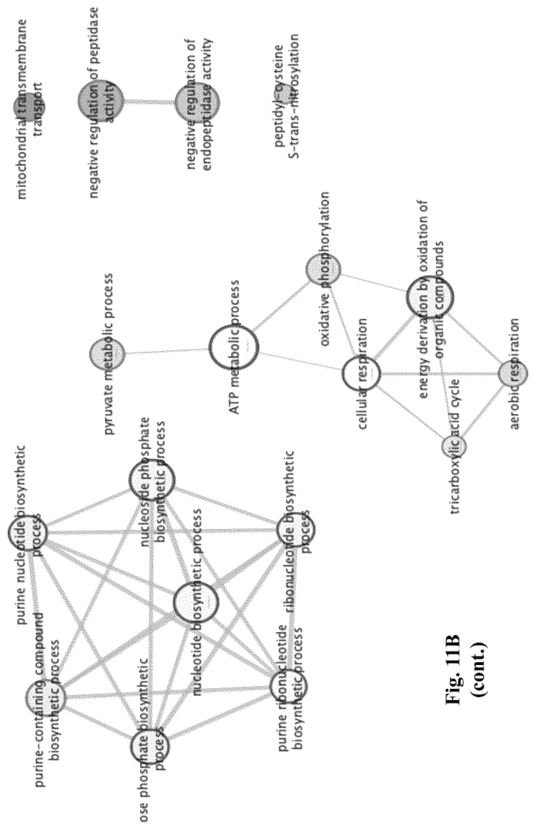

FIGS. 11A-B show aspects of identifying cardiac targets. FIG. 11A shows a set up for mass spectrometry characterization of autoantibodies in patients. FIG. 11A shows proteomics data showing specific autoantibody pattern for subclinical myocarditis patients.

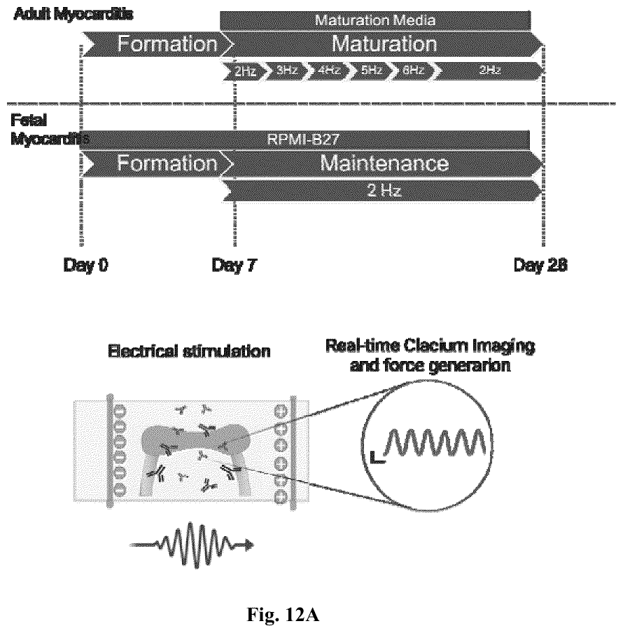

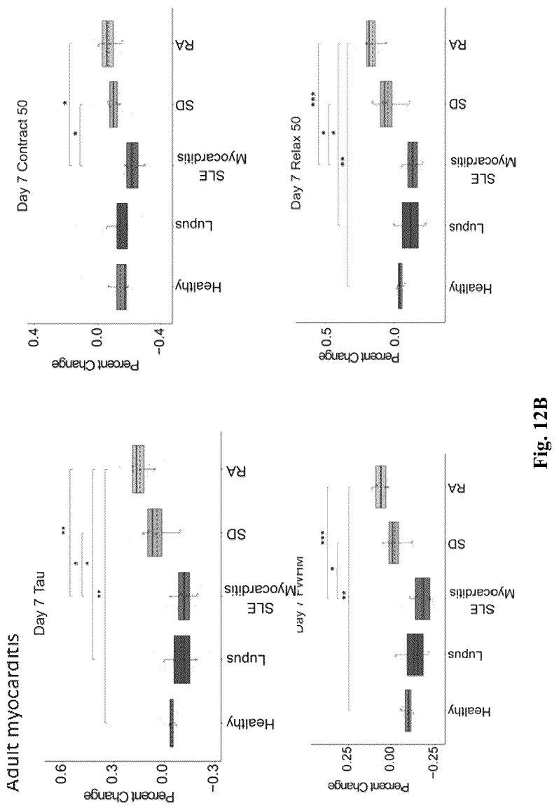

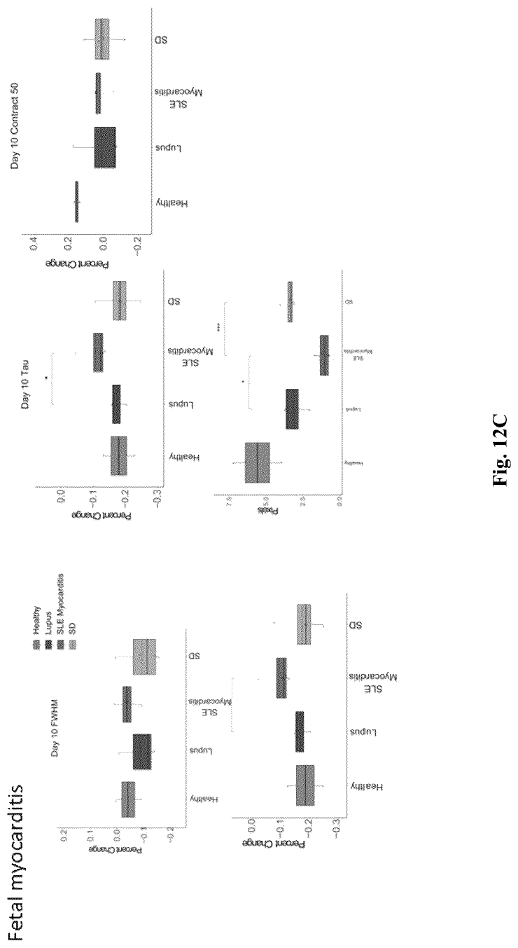

FIGS. 12A-C show how engineered cardiac tissue performance correlates with patient diagnosis in adult and fetal myocarditis models. FIG. 12A shows the experimental set up for culturing cardiac tissue models together with patient serum in two different tissue models. (FIGS. 12B,C) Prolonged calcium transients in tissues cultured with myocarditis autoantibodies in both adult and fetal models of myocarditis.

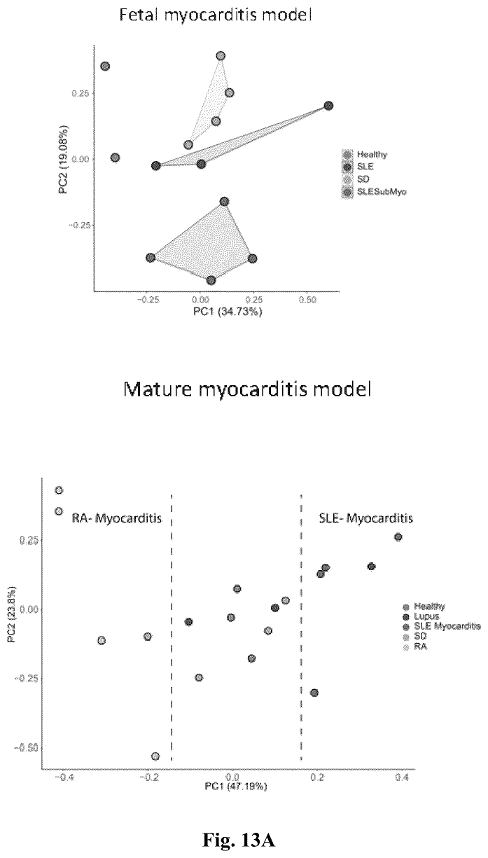

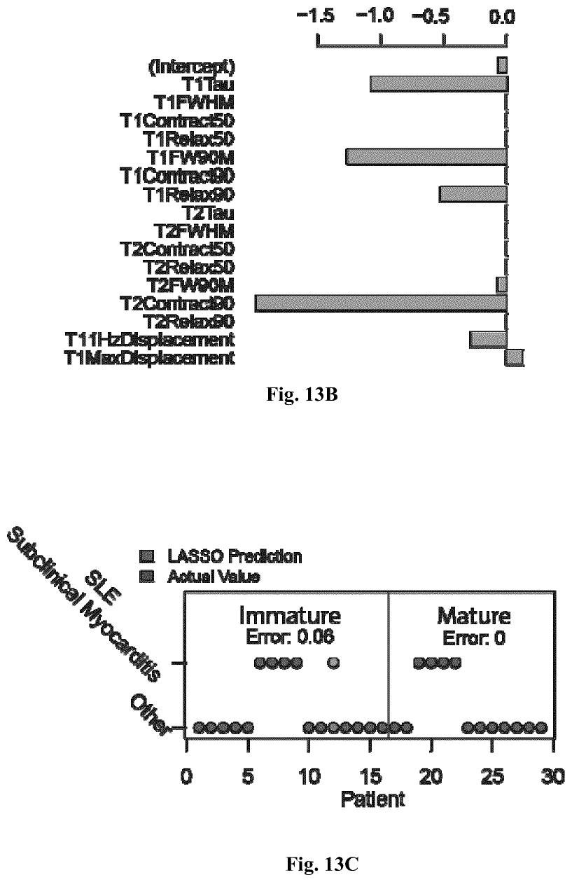

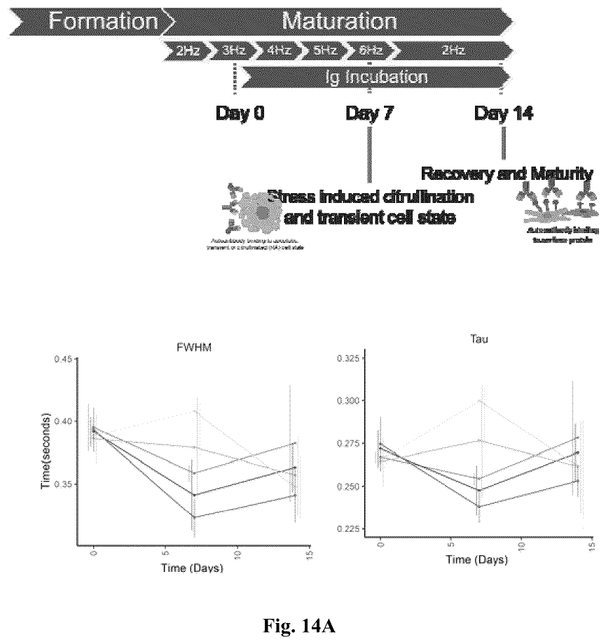

FIGS. 13A-C show how combining calcium handling and force generation scores improves in vitro patient diagnosis. FIG. 13A shows that PCA techniques can be used to segregate patient groups using calcium and force functional data. This PCA can be used to figure out if an unknown patient belongs to the myocarditis group. FIG. 13B shows a multiple regression predictive model can be used to diagnose patients. Coefficients determine the weight of each functional metric in the model. FIG. 13C shows a multiple regression predictive model to diagnose patients with myocarditis using functional metrics (calcium and Force).

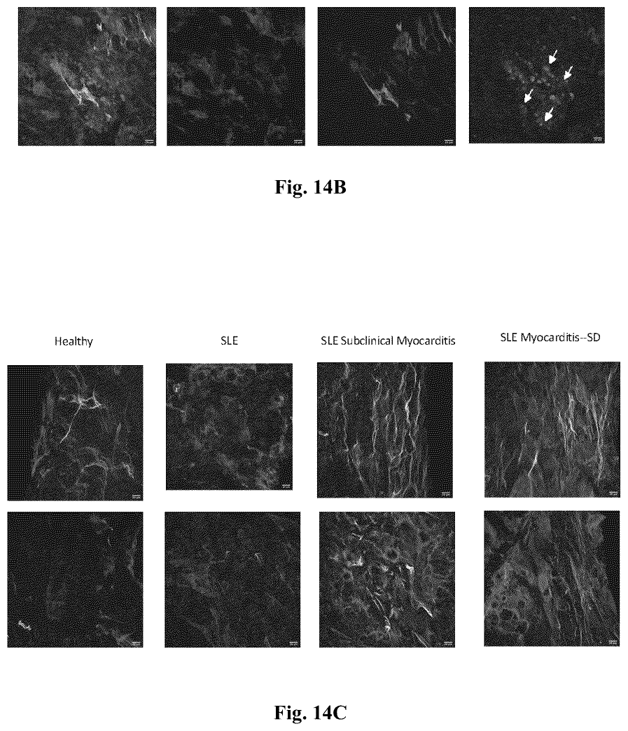

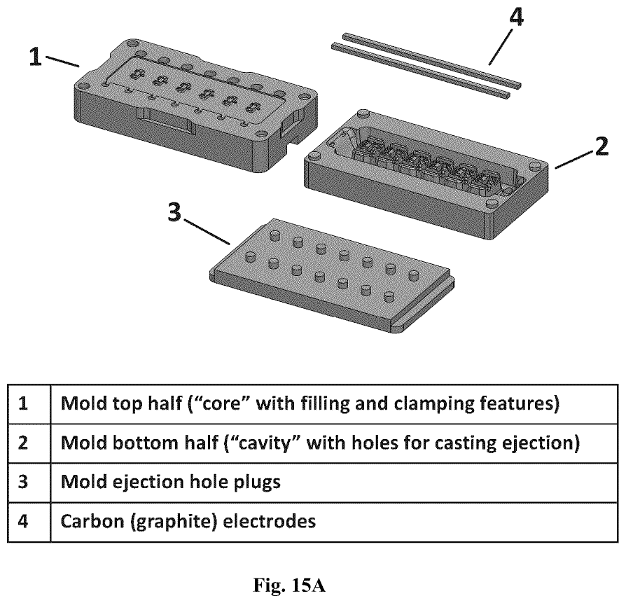

FIG. 14A shows that tissue stress leads to more pronounced differences between myocarditis patients and controls. FIG. 14B shows how autoantibodies bind to apoptotic blebs. FIG. 14C shows functional changes in cardiac tissues cultured with autoantibodies from myocarditis patients is accompanied by structural changes as shown by increased fibrosis (yellow) in staining. Cardiomyocyes (red), fibroblasts (yellow).

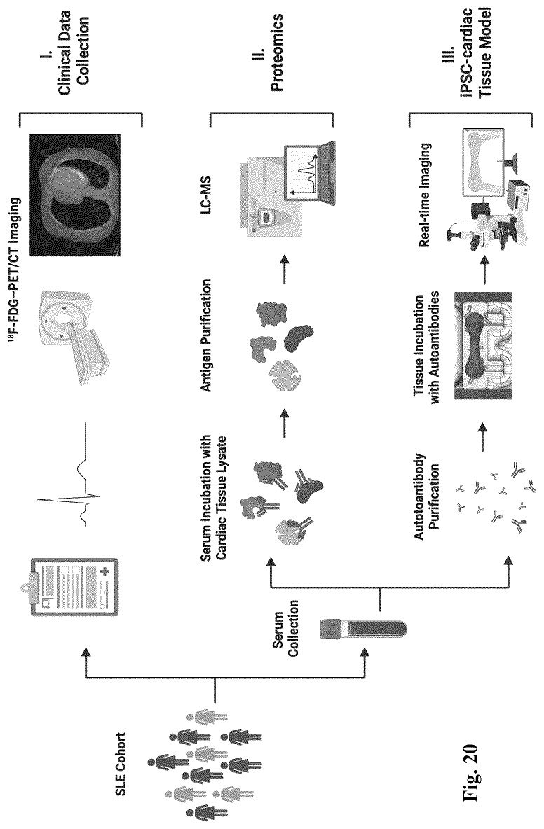

We report here a study of the cohort of SLE patients with extensively characterized cardiovascular status, including the use of ¹⁸F-fluorodeoxyglucose-positron emission tomography/computed tomography (¹⁸F-FDG-PET/CT) to quantify the extent of myocardial inflammation. We collected serum from SLE patients with myocarditis (n=9), without myocarditis (n=3) and from healthy controls without SLE or myocarditis (n=3) and identified their distinct autoantigen specificity profiles by liquid chromatography-mass spectrometry (LC-MS). We purified autoantibodies from the patients' sera and incubated them with engineered cardiac tissues in a custom bioreactor to investigate the resulting effects on calcium handling and tissue contractility in real-time (FIG. 20). Clinical data and serum samples were collected from patients in a longitudinal SLE patient cohort. (I) Clinical data included comprehensive SLE and heart disease characterization. ¹⁸F-FDG-PET/CT scans were performed to determine the presence and severity of myocarditis. Representative ¹⁸FDG-PET/CT image showing diffuse myocardial FDG uptake (white arrow heads) on a transverse view of the heart of a patient with SLE and concomitant myocarditis. (II) Identification of cardiac autoantigens targeted by autoantibodies present in patients' serum, by LC-MS. (III) Autoantibodies were purified from the patients' serum and cultivated with human cardiac tissues. Real-time calcium and brightfield imaging were taken to measure tissue calcium handling and contractility, respectively, over time.

### EXAMPLES

### Materials and Methods

### Reactor Design

The reactors and molds were designed in SolidWorks (Dassault Systemes, Vélizy-Villacoublay, France). The bending of the pillars was simulated with COMSOL Multiphysics (COMSOL, Inc., Stockholm, Sweden) to estimate the displacement of each portion of the pillars throughout a bending cycle.

### Reactor Fabrication

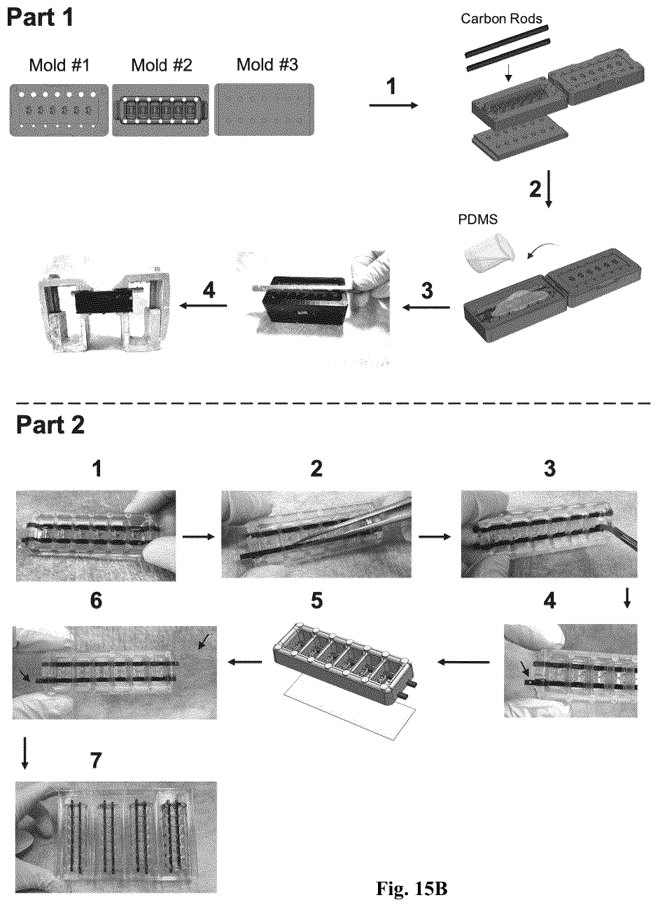

A computer numerical control (CNC) milling machine was used to make 3 sets of molds out of Delran to generate one reactor (FIGS. 15A and 15B). The molds were deburred and subsequently casted with PDMS (Dow Corning Sylgard 184) three times to clear debris before initial use. Before casting, the pillar spaces in the molds were cleaned with pressurized air to ensure proper pillar formation. Metal tools should not be used in this area to avoid scratching. To fabricate reactors, parts #1 and and #2 were assembled and carbon rods (Ohio Carbon Black, Squares & Plates, AR-14, Semi precision, Saw-Cut X=2.63 Y=0.0790 Z=0.0590) were placed into mold part #2. PDMS (10:1 ratio of base: curing agent) was mixed thoroughly, degassed, and casted into part #2. An additional degassing stage was performed for 45 minutes, or until no more bubbles were visible (if applicable a vacuum that can reach a pressure of (−635) to (−760) mmHg may be used). Next, the top of mold #2 was covered with mold #3. The assembled molds were then clamped with the hex screw in place, topped off with PDMS, and placed into a 65° C. oven overnight. Reactors were excavated with a flat tool, by gently separating the sides of the reactor from the mold many times until the reactor slid out. PDMS was cut off the ends of the reactor and the PDMS film on the rods within the wells was removed with a tweezer and scalpel. A 1/32″ hole was drilled into the ends of the rods. The reactors were sonicated with 1% Tween-20 in distilled water for 1 hour. Reactors were then rinsed thoroughly with distilled water and allowed to dry in a 65° C. oven overnight. Simultaneously slides were cut to 25 mm×60 mm. Reactors were bonded to slides with 5 mbarr Oxygen Plasma treatment for 30 seconds. To ensure a tight seal between the reactor and the glass slide a small paint brush was used to apply PDMS to the ends of the reactor with the slide and placed in a 65° C. oven overnight. A platinum wire (Superpure Chemical, cat. no. 2805) was threaded through the drilled holes and wrapped around the carbon rods. As mentioned above, ideally, a large batch of reactors should be fabricated at the same time to minimize batch-to-batch variability.

### Pillar Force-Displacement Analysis

The force required to displace the pillar was determined using a microscale mechanical tester, Microtester (CellScale). A 0.4064 mm diameter circular tungsten microbeam with platen (1 mm×1mm) was used to displace the pillar head. Before the test, the platform was fixed on the testing stage with clamps. The probe tip with platen was placed adjacent to the pillar head without contact and gradually moved towards the center of the platform at a velocity of 8.5 μm/s. The tip displaced the pillar head and applied the force perpendicular to the original pillar position. All platforms were fabricated according to the protocol mentioned previously. Four to six pillars were tested in each millipillar culture platform and four batches of millipillar culture platforms were included. The experimental data were fit into a linear equation, generating a force-displacement calibration curve with a coefficient that can be used to calculate active forces and passive tensions based on the position of the pillar head during the experiment.

### Electrical Stimulation

A custom electrical stimulator was designed to work within the open source Arduino software and hardware environment. Briefly, the circuit consists of an Arduino Uno Rev3 microcontroller development board (Arduino, cat. no. A000066), a digital potentiometer (Microchip Technology, cat. no. MCP42100-I/P), a power operational amplifier (Texas Instruments, cat. no. TLV4112IP), two dual channel H-bridge motor drivers (Pololu Robotics, cat. no. 2135), and a series of 1 ohm test resistors. The microcontroller sets the stimulation voltage by adjusting the resistance of the digital potentiometer, which is placed between +5V and ground in the circuit. The wiper from the digital potentiometer connects to the power op-amp in a unity gain configuration such that the output of the op-amp maintains the specified voltage but with the capability to supply a much greater current (˜300 mA). This output powers the motor drivers and provides the current for stimulation. Each motor driver channel is controlled by two digital outputs from the microcontroller using the driver's PHASE/ENABLE mode to generate biphasic pulses at +/− the specified stimulation voltage supplied to the drivers. The frequency, duration, and phase offset of these pulses are specified in the Arduino code and can be easily customized. Monophasic stimulation can also be selected instead of biphasic. Each output channel, of which there are four total, can operate independently at a unique frequency, but all channels share a common output voltage. Due to the incorporation of field-effect transistors within the motor drivers, there will be a slight voltage drop across the motor drivers that varies real-timearly with the output current. We recommend measuring the output voltage after connection to the bioreactor and adjusting if necessary.

### Cardiomyocyte Differentiation

hiPSCs were obtained through material transfer agreements from B. Conklin, Gladstone Institute (WTC11 and GCaMP6f-WTC11 lines) and Columbia University's Stem Cell Core (BS2 line).

Cardiomyocytes were differentiated from iPSCs (WTC, WTC11-GCamp6f and BS2) as previously described²¹. On Day 10, RPMI-no glucose (Life Technologies, cat. no. 11879020) supplemented with B27 (Thermo Fisher Scientific, cat. no. 17504044) and 213 μg/ml ascorbic acid (Sigma-Aldrich, cat. no. A445), was used to purify the iPSC-CMs population and eliminate potential contaminating mesodermal and endodermal populations. Starvation media was replaced on day 13 and returned to RPMI-B27 media supplemented with 213 μg/ml ascorbic acid until day 16. On day 17 cells were pretreated with rock inhibitor (y-27632 dihydrochloride, 5 uM) for 4 hours before dissociation. Cells were dissociated by enzyme digestion with collagenase type II (95U/mL; Worthington, cat. no. LS004176) and pancreatin (0.6 mg/mL; Sigma-Aldrich, cat. no. P7545) in dissociation buffer (Glucose (5.5 mM), CaCl2·2H20 (1.8 mM), KCl (5.36 mM), MgSO4·7H2O (0.81 mM), NaCl (0.1 M), NaHCO3 (0.44 mM), NaH2PO4 (0.9 mM)) on a shaker in a 37° C. incubator. After 10 minutes a 5 ml pipette (or cell scraper) can be used to gently triturate the cells and lift them off the plate. Cells were placed back in the incubator for 10 minutes until cells were dissociated into single cells. With a 5 ml pipette, the cardiac suspension was triturated and added to a 50 mL conical tube. Cells were gently triturated again to form a homogenous suspension. One volume of RPMI-B27 media was added to the tube and the cells were spun down at 1200 RPM for 5 minutes. Cell purity of at least 85% is required to ensure reproducibility. Therefore, flow cytometry for cTNT+(BD BioSciences cat. no 565744) was performed prior to cell use for tissue fabrication. Cells can be frozen in freezing media (CryoStor® CS10, Stem Cell Technologies, cat. no. 07955) at a concentration of 5-10 million/mL or can be used right away. If cells were thawed, media was added drop by drop for 60-90 seconds, filled to an appropriate volume slowly, and then spun at room temperature at 100×g. Cells were kept on ice for the tissue making process.

### Fibroblasts

Primary Human cardiac fibroblasts (NHCF-V; Lonza, cat. no. CC-2904) and dermal fibroblasts (NHDFs; Lonza, cat. no. CC-2509 were obtained from LONZA and cultured according to manufacturer's recommendation. iPS-CFs were differentiated according to previously described protocol.

### Generation and Culture of Engineered Cardiac Tissues

Either thawed or fresh cells were resuspended in RPMI-B27 media to form a cell mixture with a previously optimized ratio of fibroblasts (25%) to cardiomyocytes (75%). Precise cardiomyocyte purity is as determined by flow cytometry for cTnT (BD BioSciences cat. no 565744. For fibrin tissues, the cell mixture was subsequently resuspended in fibrinogen by mixing 33 mg/ml human fibrinogen (Sigma-Aldrich, cat. no. F3879) to a final fibrinogen concentration of 5 mg/mL. The volume of each individual tissue is 15 μL and contains 550,000 cells (366,666 cells/μL). When calculating the amount of fibrinogen to add, the volume of the cell pellet and the volume of the thrombin solution is taken into account. 3 μL of thrombin (2U/mL) were added to each well. Immediately after 12 μL of fibrinogen-cells solution was dispensed into the well, and quickly spread over the entire well with a pipet tip. Tissues were placed in a 37° C. for 15-20 minutes to allow gelation in the well. If tissues in more than one reactor are formed, fibrin-cell suspensions should be kept homogenous by frequent mixing, without introducing bubbles. Pipette tips should be replaced after seeding each tissue to prevent residual thrombin crosslinking the fibrin-cells stock suspension. A collagen I gel was prepared according to manufacturer's protocol (Advanced Biomatrix, Cat. no. 5279) and mixed with cells for a final concentration of 4 mg/mL Collagen. Collagen tissues were prepared by simply adding 15 ul of the cell-gel suspension to each well.

400 ul of RPMI-B27 (with 213 μg/ml ascorbic acid and, 10 uM Rock inhibitor and 5 mg/ml aminocaproic acid (Sigma-Aldrich A7824, aminocaproic acid is only necessary for fibrinogen tissues) was added to the reactors. After 1 hour a 26 gauge needle was used to detach tissues from the walls of the reactor. 24 hours later, tissues were detached again and the media was changed to RPMI-B27 with 213 μg/ml ascorbic acid and 5 mg/ml 6-aminocaproic acid. The media was changed every three days for 6 days. On Day 7, 6-aminocaproic acid was removed from the media and electrical stimulation was started with the stimulator using a 5V/cm biphasic pulse (2 ms pulse length, 1 ms per phase) at 2 Hz. Tissues were either paced at 2 Hz continuously, or following the previously reported ramp stimulation regimen [Nature paper]. During the ramp stimulation regimen, the frequency started at 2 Hz and was increased everyday by one-third Hz until reaching 6 Hz. 6 Hz stimulation was maintained for three days, after which stimulation frequency was reduced to 2 Hz and maintained until endpoint analysis at day 21. Stimulation voltage and pulse duration were not modified during the stimulation regimen. Media was changed every other day.

### IF and Histology

Whole mount engineered cardiac tissues were fixed and permeabilized in 100% cold methanol for 10 min, washed three times in PBS , and then blocked for 1 h at room temperature in PBS with 2% FBS. For tissue sections, tissues were fixed in 4% PFA for 30 min and washed three times in PBS. Whole tissues were placed flat in the bottom of 15 mm square disposable histology base molds and encapsulated in 1 mL molten Histogel (Fisher Scientific, cat. no. 22-110-678). These Histogel blocks were cooled according to the manufacturer's protocol and then fixed a second time with 4% PFA for 30 minutes followed by three washes with PBS. Fixed Histogel blocks were paraffin-embedded and cut into 5-μm-thick sections. Paraffin sections underwent heat mediated antigen retrieval in a pH 6 sodium citrate buffer, permeabilized with 0.01% triton in PBS and then placed into the blocking buffer using 10% FBS. After blocking, the tissues were incubated with primary mouse anti-α-sarcomeric actinin antibody (1:750, Sigma-Aldrich, cat. no. A7811), anti-cardiac troponin T (cTnT, 1:100; Thermo Fisher Scientific, cat. no. MS-295-P1), and vimentin (abcam, cat. no. ab92547) washed three times, and incubated for 1 hr with secondaries antibodies. For nuclei detection, the tissues were washed and subsequently incubated with NucBlue (Molecular Probes, R37606). Samples were visualized using a scanning laser confocal microscope (Nikon Eclipse Ti) or a DMi8 microscope (Leica Microsystems).

### Calcium Imaging

To make tissues with an endogenous marker of cytosolic calcium, we used iCMs derived from previously engineered WTC11-GCaMP6f iPSCs that contain a constitutively expressed GCaMP6f calcium-responsive fluorescent protein inserted into a single allele of the AAVS1 safe harbor locus. These cells were a gift from Bruce Conklin. The incorporation of these GCaMP6f cells allows real-time visualization of calcium transients without the need for additional dyes. Tissues were imaged using a sCMOS camera (Zyla 4.2, Andor Technology) connected to an inverted fluorescence microscope with a standard GFP filter set (Olympus IX-81). To assess calcium transients in tissues made with non-GCaMP cell lines, cardiac tissues can be incubated with 10 μM fluo-4 AM (Invitrogen, cat. no. F14201) and 0.1% Pluronic F-127 (Sigma-Aldrich) for 45 min at 37° C.

Cardiac tissues were stimulated once with the analysis stimulation regimen to acclimate all tissues for measurement. Videos were then acquired at 20 fps for 4600 or 300 frames, respectively, using the 488 fluorescent channel.

### Measuring Brightfield Contractions

Cardiac tissues were stimulated once with the FFR custom program to acclimate all tissues for measurement. Videos were then acquired at 20 fps for 4800 frames using a custom program to stimulate cardiac tissues from 0.5 Hz to 4 Hz.

### Analyzing Calcium Signal

A custom Python script was developed to average the pixel intensities for each frame. This transient was then corrected for fluorescent decay. The SDRR, Tau, FWHM, FW90M, Contract90, Contract50, Relax50, and the Relax90 were calculated for every transient (Supplementary Figure XX). An average of every transient over 15 seconds was calculated and represented in a table. When calculating ET/MCR, the custom program provides traces for each stimulation frequency and presents when tissues begin to beat out of sync from stimulation. Manual inspection of each trace is recommended due to the sensitivity of the program to cardiac tissue abnormalities (spiral waves, etc.).

### Analyzing Force Generation

A custom Python script was developed to track the motion of the pillar heads and then the force was calculated by multiplying the displacement of the pillars with the coefficient determined from the force-displacement curve generated for the PDMS pillars. This script uses the correlation tracker class from the open-source dlib C++library (https://github.com/davisking/dlib) to determine the location of the pillar heads in each frame based on initial bounding boxes placed around the pillar heads in the first frame by the user. The script uses the location of the pillar heads to determine the total deflection of the pillar from their equilibrium position without any force applied. The dlib correlation tracker is an implementation of a previously published object-tracking algorithm that uses a cosine correlation filter applied to a histogram of ordered gradients (HOG) feature descriptor for each frame in the recording.

### Statistical Analysis

Statistical analyses were performed using GraphPad prism. Single comparisons of data were assumed to follow a normal distribution and assessed using a two-tailed paired Student's t-test to determine statistical significance. A p-value of <0.05 was considered statistically significant. *P<0.05, **p<0.01 ***p=0.001 ****p<0.0001.

### Clinical Characteristics of SLE Patients

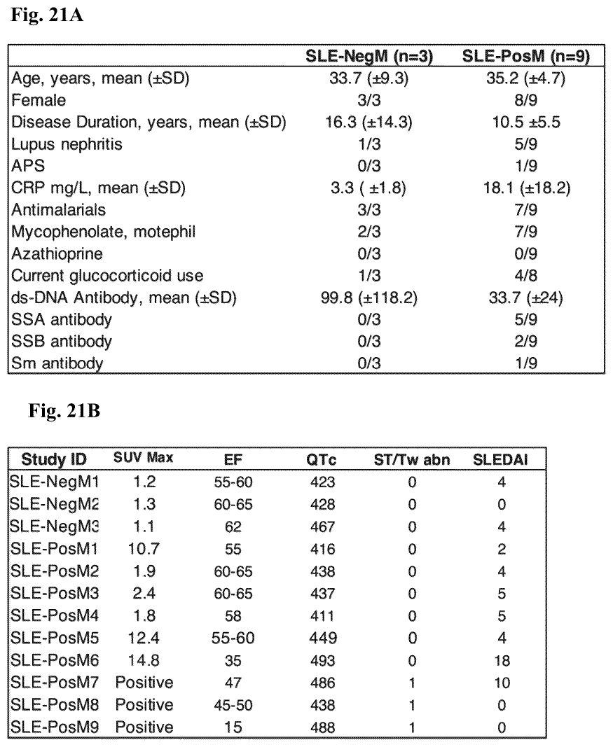

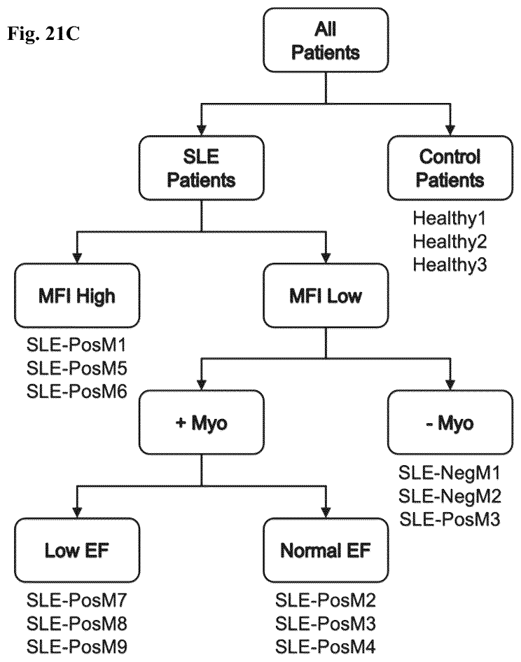

Clinical data for the cohort of SLE patients are summarized in FIGS. 21A, 21B and 2C. To determine the presence and severity of myocarditis, all patients were evaluated by cardiac ¹⁸F-FDG-PET/CT. Myocardial FDG uptake was first assessed qualitatively and then quantitatively in a subset of patients by standardized uptake values (SUV). Grouped clinical characterization of SLE patients without myocarditis (SLE-NegM) and SLE patients with myocarditis (SLE-PosM). (b) Selected clinical measures for the patients in the study. (c) Patient groupings used in subsequent analysesPatients were classified according to the measured FDG-uptake levels into SLE patients without myocarditis (SLE-NegM, SUVmax<1.5; n=3) (FIG. 16A) and SLE patients with myocarditis (SLE-PosM, SUVmax>1.5; n=9). Among the patients with myocarditis, 5 had preserved systolic function with LVEF>50% and 4 had reduced LVEF<50%. All SLE patients without myocarditis had LVEF>50% (FIG. 21B). Patient SLE disease activity index (SLEDAI) scores ranged from 0 to 18. None of the included patients had detectible levels of serum troponin based on a high sensitivity cardiac troponin assay. Additionally, none of the patients had diabetes or were smokers during the study at the time of imaging and serum collection. Patient groupings used in subsequent analysis are depicted in FIG. 21C.

### Patient-Derived Autoantibody Profiles

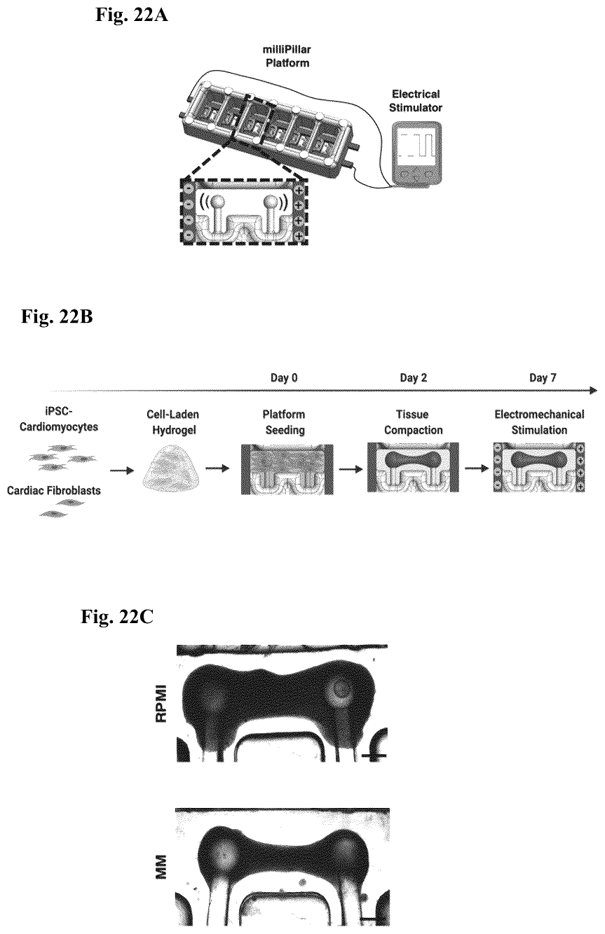

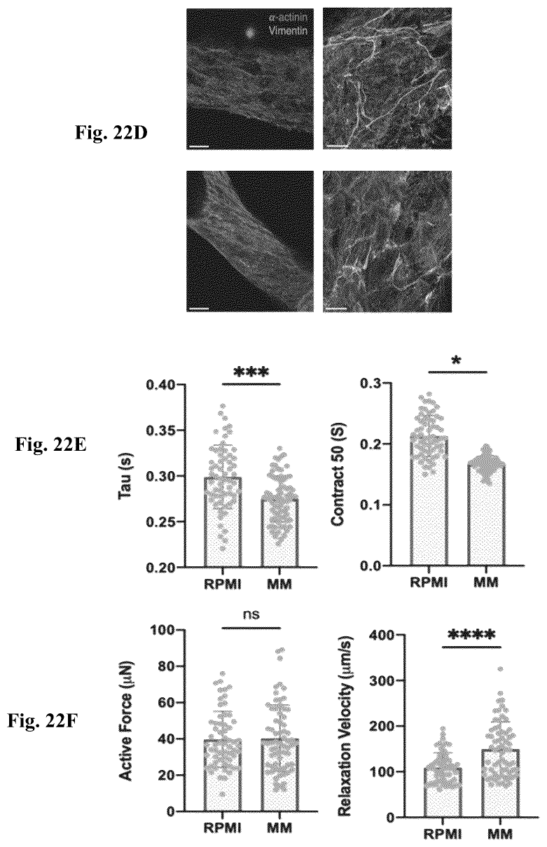

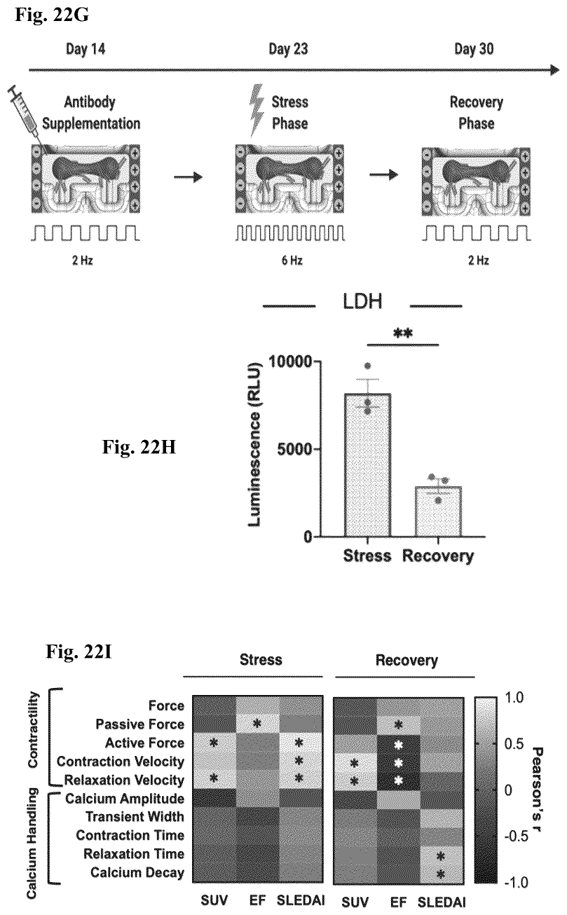

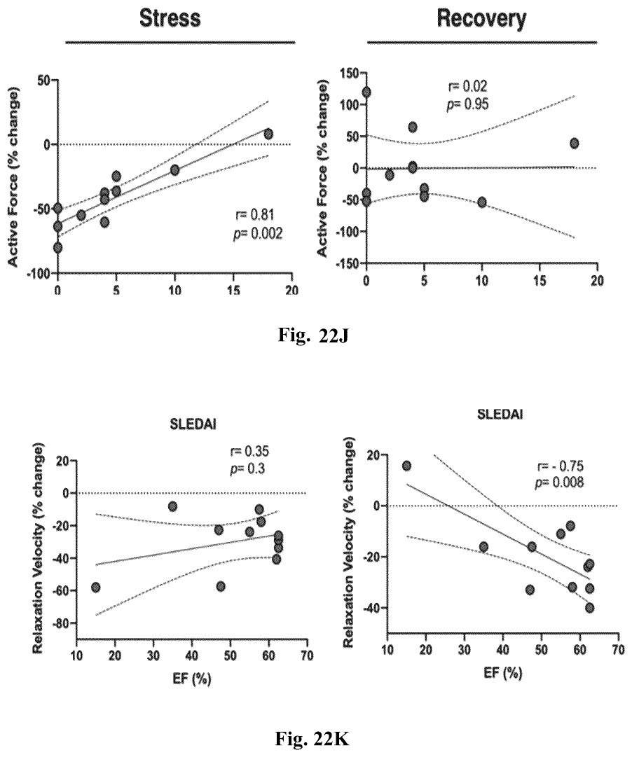

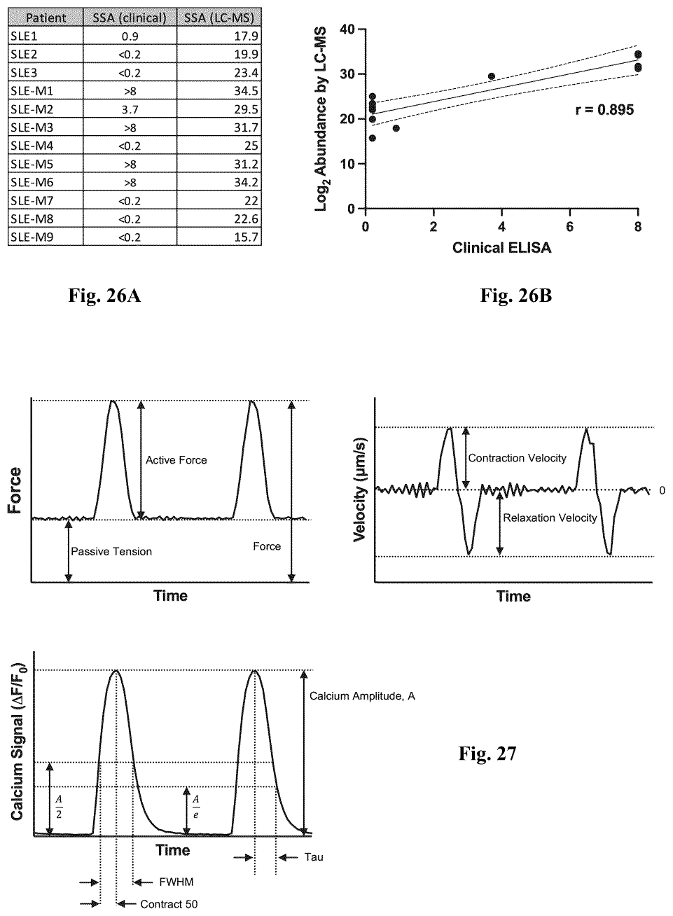

SLE autoantibodies profiles have been widely studied, however their role in the manifestation of adult SLE-myocarditis remains unclear. We thus sought to first identify and characterize the specific autoantibody profiles for all patients and healthy controls, by identifying their cognate autoantigens by LC-MS (FIGS. 22A-K). The accuracy of autoantigen detection was confirmed by a strong correlation of SSA measurements in the engineered cardiac tissue model using LC-MS to clinical SSA measurements by ELISA (FIGS. 26A,B). LC-MS data analysis revealed highly variable profiles of autoantibodies targeting cardiac tissues among patients. The identified antigens included both the well-characterized SLE targets (such as Ro52, Ro60, and SSB) and cardiac targets that have not been previously identified in SLE.

Engineered human cardiac tissues displayed structural and functional maturation. We used the recently developed milliPillar platform describrd herein for the engineering, maturation, and functional evaluation of human cardiac tissues and to study the role of patient's autoantibodies in the pathogenesis of SLE-myocarditis.

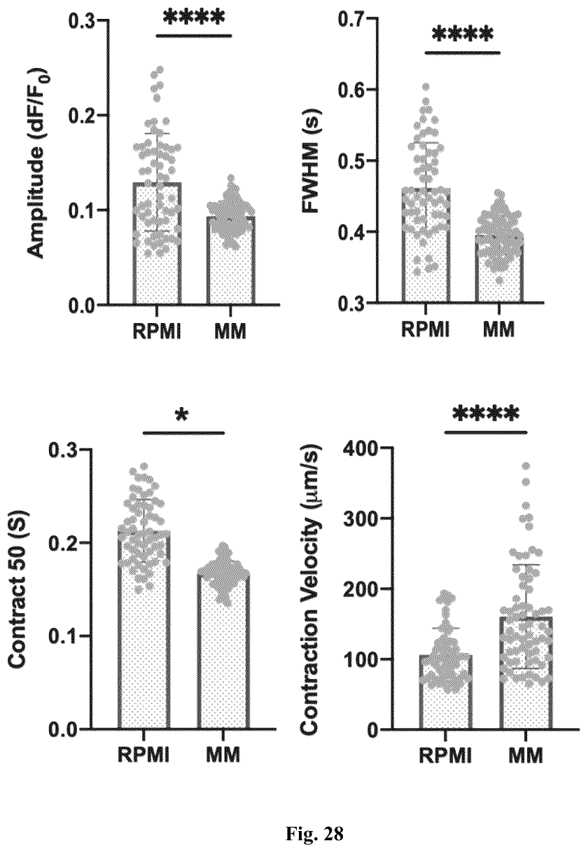

FIG. 22A shows schematics of the human cardiac tissue platform, termed milliPillar. The platform contains 6 individual chambers, each with two flexible pillars between two parallel electrodes connected to an electrical stimulator. FIG. 22B shows that cardiac tissues were fabricated by resuspending human iPSC-derived cardiomyocytes and primary cardiac fibroblasts in fibrin hydrogels that compacted around the pillars and were electrically stimulated. The platform supports six individual micro-sized tissues, each attached to two flexible pillars. FIGS. 22C and 22D show representative brightfield images (22C) and immunofluorescence images (22D) of cardiac tissues cultured in RPMI medium or MM medium and stimulated at 2 Hz frequency for 7 days. FIGS. 22E-F show that electromechanically stimulated engineered cardiac tissues cultured in MM medium demonstrated improved calcium handling (22E) and contractility (22F) when compared to tissues cultured in RPMI medium (RPMI: n=61 tissues, MM: n=75 tissues; two-sided unpaired Student's t-test). FIG. 22G shows that on day 14, tissues were supplemented with autoantibodies purified from SLE patients, and electrical stimulation was gradually increased from 2 Hz to supraphysiological frequency of 6 Hz over a 7-day period (stress phase). The frequency was then decreased to 2 Hz and maintained at that level until day 30 (recovery phase). Brightfield and calcium images were recorded on day 14, 23, and 30 to evaluate tissue function before, during, and after stress, respectively. FIG. 22H shows that supernatant LDH quantification showed enhanced cytotoxicity during the stress phase when compared to the recovery phase (n=3 tissues; two-sided paired Student's t-test). FIG. 22I depicts heat maps representing Pearson correlation coefficients show correlations between in vitro cardiac tissue performance and clinical measures of disease severity (SUV, EF, SLEDAI) during stress and recovery phases. FIGS. 22J-K show linear regression analysis of active force generation by cardiac tissues against corresponding patient SLEDAI (22J), and cardiac tissue relaxation velocities against corresponding patient EF (22K) revealed distinct patient-specific autoantibody effects on tissue function during stress and recovery. Scale bars; 100 μm (left) and 20 μm (right). SUV: standardized uptake value; EF: Ejection fraction; SLEDAI: Systemic Lupus Erythematosus Disease Activity Index; MM: maturation medium. Data are presented as mean ±SEM. *p<0.05, **p<0.005, ***p<0.0005, **** p<0.0001. Automated imaging and data analysis pipelines were used to characterize the contractility and calcium handling of the tissues throughout culture (FIG. 27). Tissues were cultured in specialized metabolic maturation medium (MM) to shift cell metabolism from anerobic glycolysis toward fatty acid oxidation and subjected to electrical stimulation to induce macroscopic contractions. Tissue maturation by the combined use of maturation medium and electromechanical conditioning promoted tissue compaction (FIG. 22C), cell alignment, α-actinin striations (FIG. 22D), calcium handling and tissue contractility (FIGS. 22E, 22F and 28).

In vitro function of cardiac tissues cultured with patient autoantibodies correlated with the clinical measures of disease severity in a stress-dependent manner. We previously implemented electrical stimulation at frequencies increasing from 2 to 6 Hz (120 to 360 beats per minute) over two weeks to promote tissue maturation. Here we used a modified version of this protocol to induce cardiac stress and inflammatory conditions by electrical stimulation at supraphysiological frequencies (6 Hz) and then allowed the tissues to recover at a frequency of 2 Hz for another week (FIG. 22G). We confirmed transient tissue stress conditions by significantly higher levels of lactate dehydrogenase (LDH) in supernatant during the stress phase compared to recovery phase (p=0.004; FIG. 22H).

We then investigated whether the functional responses of tissues cultured with patient autoantibodies would correlate to patient-specific clinical features of the disease. Autoantibodies isolated from SLE patients with and without myocarditis were added to the culture medium prior to the initiation of stress and were cultured with the tissues throughout the stress and recovery phases (FIG. 22G). Each tissue was cultured with autoantibodies from a single patient.

Functional metrics were obtained via video analysis. To decrease tissue-to-tissue variability, all metrics were measured as percent change from baseline. By linear regression between the functional metrics of tissues cultured with each patient's autoantibodies and the corresponding clinical measures for the same patient, we found that tissue contractility and calcium handling strongly correlated with the severity of myocardial inflammation (SUV), systolic function (LVEF), and severity of SLE disease (SLEDAI) (FIG. 22I).

Each clinical measure had a different correlation to the functional parameters, indicating that distinct autoantibody patterns lead to differential effects on tissue function. Moreover, we observed different correlation patterns for each clinical measure in the disease and recovery phases (FIGS. 22J-K). For example, while SLEDAI levels strongly correlated with tissue contractility during the stress phase, these correlations were lost during the recovery phase, where strong correlations were observed with calcium handling measurements. In addition, while EF strongly correlated with passive force during the stress phase, during the recovery phase it correlated with active force, contraction velocity, and relaxation velocity as well.

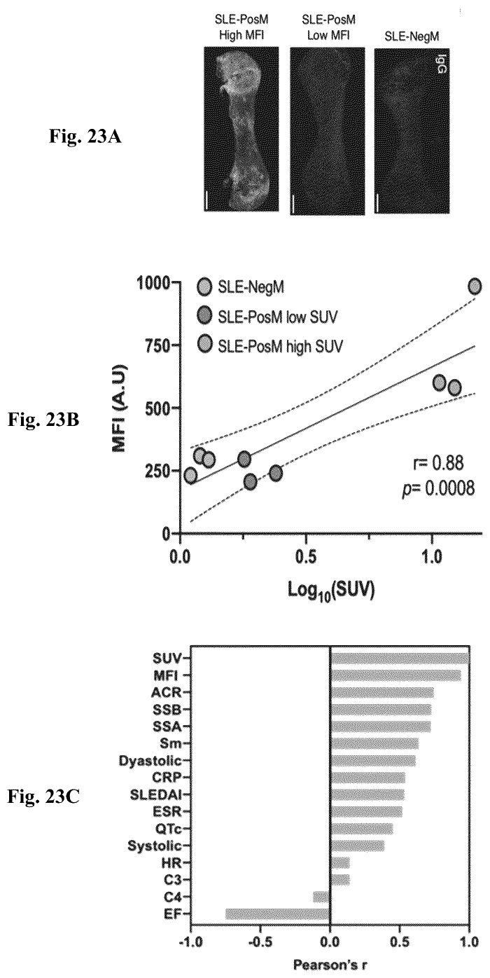

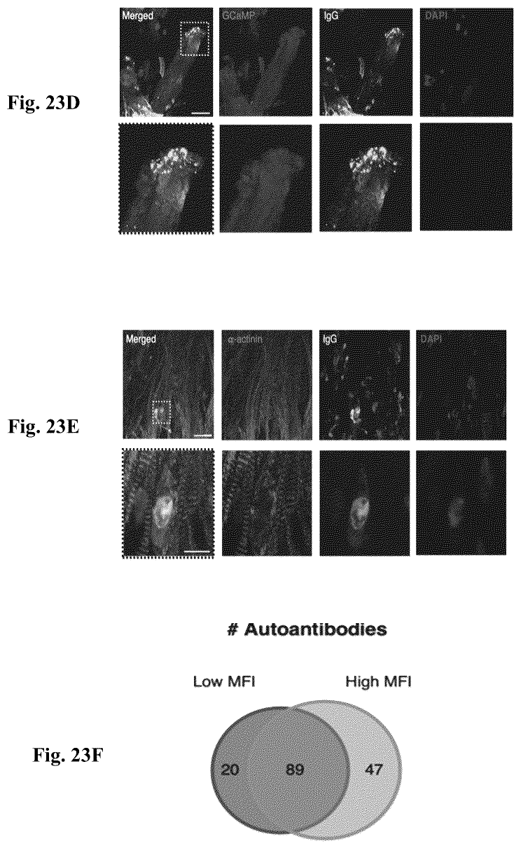

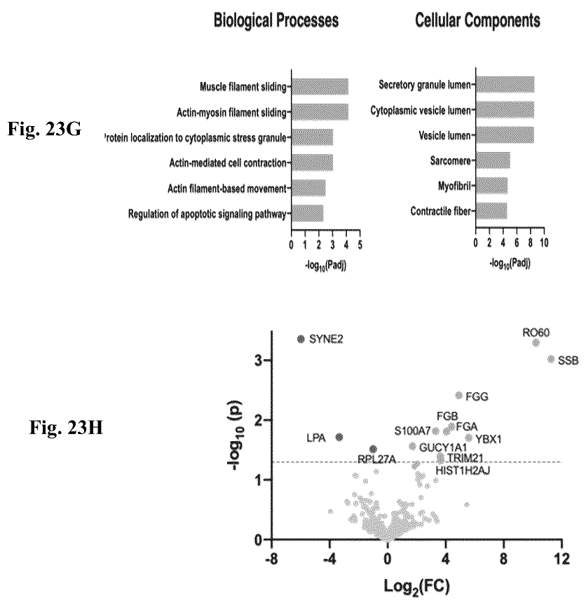

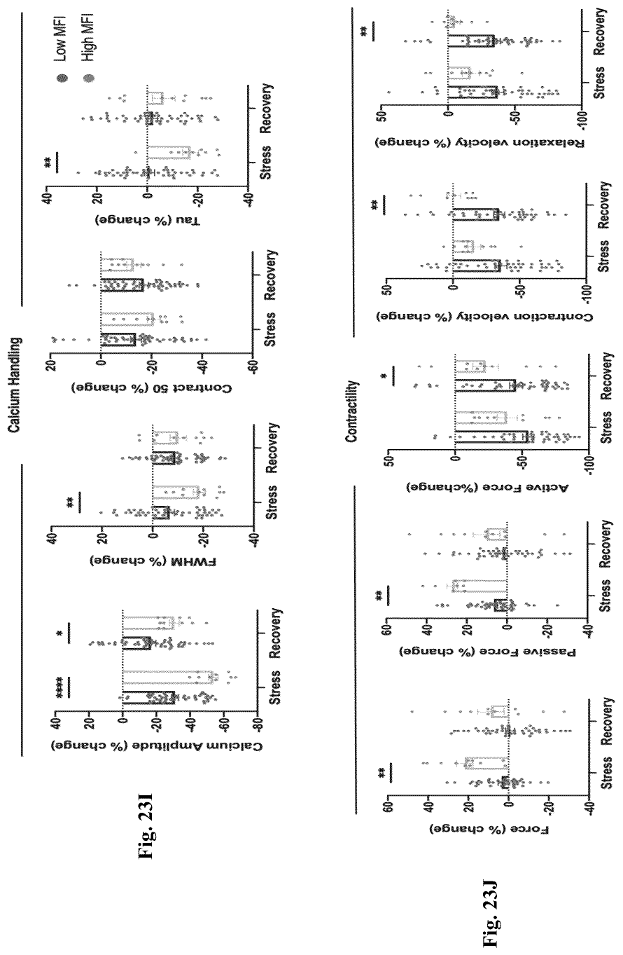

FIGS. 23A-J show aspects of determining that SLE patients with high levels of myocardial inflammation have high levels of autoantibodies binding to cardiac tissues under stressed conditions, leading to altered cardiac tissue functionality. FIG. 23A shows representative indirect immunofluorescent images of cardiac tissues cultured with purified autoantibodies demonstrated enhanced autoantibody binding (high MFI) in SLE patients with severe myocarditis (high SUV). FIG. 23B shows linear regression analysis revealed strong correlation between tissue MFI measurements and SLE patients with (PosM) and without (NegM)) myocarditis (determined by SUV levels). Black solid line: the best-fit line; black dashed lines: the 95% confidence intervals. Pearson correlation analysis was used to assess significance and demonstrated that in vitro MFI measurements correlate more strongly with clinical FDG uptake (SUV) than with any other clinical measure. FIGS. 23D-E show representative immunofluorescence images of cardiac tissues cultured with autoantibodies from SLE high MFI patients that show enhanced binding of autoantibodies to apoptotic blebs on the surface of the cells (23D) and the stressed cardiomyocytes (23E). FIG. 23F shows a Venn diagram representing the number of autoantigens identified in SLE patients with high MFI levels relative to SLE patients with low MFI levels. FIG. 23G shows top biological processes and cellular compartment GO terms associated with autoantibodies present in patients with high MFI levels are related to cardiac contractility and cellular stress. FIG. 23H shows volcano plots demonstrated differentially targeted autoantigens comparing SLE patients with high MFI levels (right) and low MFI levels (left), determined on the basis of p≤0.05 and log2(fold change) ≥1 (low MFI: n=3 patients, high MFI: n=9 patients; two-sided Student's t-test). FIG. 23I shows that calcium handling analysis revealed significant differences in calcium amplitude and τ decay between the low and high MFI groups during the stress phase (low MFI: n=49 tissues, high MFI: n=11 tissues; two-way ANOVA with Sidak's multiple comparisons test). FIG. 23J shows that contractility analysis revealed significant differences between low and high MFI patients in passive force during the stress phases, and in active force, contraction velocity, and relaxation during the recovery phase. Data are presented as mean ±SEM. *p<0.05, **p<0.005, ***p<0.0005, **** p<0.0001.

Autoantibodies from patients with high levels of myocardial inflammation demonstrated enhanced binding to stressed cardiac tissues and altered tissue function. To investigate associations between autoantibodies and specific features of SLE-myocarditis, the binding of patients' autoantibodies to cardiac tissue autoantigens was assessed by measuring mean fluorescent intensity units (MFI) via indirect immunostaining of bound immunoglobulins (FIG. 23A). SLE patients without myocarditis and a subset of patients with myocarditis had low levels of autoantibody binding to tissues (low MFI), while another subset of SLE patients with myocarditis exhibited high levels of autoantibody binding to tissues (high MFI) (FIG. 23A. Interestingly, we detected a strong correlation between patient SUV levels and MFI levels, suggesting that enhanced autoantibody binding to cardiac tissues is associated with increased myocardial inflammation (FIG. 23B). For all patients, MFI levels correlated with SUV levels more strongly than with any other clinical metrics (FIG. 23C).

We further investigated the precise tissue and cellular locations of cardiac autoantigens targeted by the autoantibodies in the immunostains of tissues from the high MFI group and found two distinct patterns. By combining indirect immunostaining with imaging of fluorescently tagged cardiomyocytes, we found that the autoantigens targeted by autoantibodies were highly concentrated in bleb-like structures on the cell surfaces, suggestive of apoptotic blebs (FIG. 23D). By combining indirect immunostaining with alpha actinin staining, which can be used to evaluate cardiomyocyte structural integrity, we found that the extent of autoantibody binding varied from cell to cell rather than being uniformly distributed throughout the tissue, indicating that only a subset of cells was targeted by the autoantibodies. Higher magnification images showed disrupted alpha-actinin striations and condensed nuclei in the positively stained cells, with staining primarily focused in the nuclei, indicating cellular damage and loss of cell membrane integrity that allows autoantibody penetration (FIG. 23E).

We next explored the identity of the autoantigens targeted by autoantibodies in the high MFI patients. We utilized detection filter analysis of the 263 autoantigens identified by LC-MS to determine the autoantibodies detected in all the samples from each group. These data revealed 47 autoantigens in all samples of the high binding group, 20 autoantigens in all samples from the low binding group, and 89 autoantigens in all samples from both groups (FIG. 23F). Gene ontology (GO) enrichment analysis of these autoantigens identified from the patients in the high MFI group pointed at biological processes involved in muscle contraction (actin-myosin filament sliding, p=6.9×10⁻⁵), stress (protein localization to cytoplasmic stress granules, p=0.00089), apoptosis (regulation of apoptotic signaling pathway, p=0.0045), cardiac contractile machinery and secreted vesicles (secretory granule lumen, p=2.4×10⁻⁹ and sarcomere, p=9.1×10⁻⁶) (FIG. 23G).

Analysis of autoantigen levels identified 10 significantly differentially targeted antigens present at higher levels in the high MFI group (FIG. 23H). Among those autoantigens we identified SSA/Ro (Ro60), Trim 21 (Ro52), and SSB/La, which are nuclear antigens and common targets of autoantibody production in SLE, especially in the context of autoimmune-mediated congenital heart block and myocarditis. These autoantigens have been shown to redistribute and cluster in surface blebs on apoptotic cardiomyocytes, as we observed on cells in the tissues.

Enhanced autoantibody binding altered tissue function. To further elucidate whether autoantibody binding levels (MFI) lead to differential effects on cardiac tissue function, we evaluated the calcium handling and contractility of tissues cultured with purified autoantibodies from patients in the low-MFI and high-MFI groups. During the stress phase, tissues cultured with autoantibodies purified from patients with high MFI exhibited significantly lower calcium amplitudes (p<0.0001), shortened calcium transients (FWHM, p=0.0045) and faster calcium uptake transients (τ decay, p=0.0014) compared to tissues cultured with autoantibodies from low MFI patients (FIG. 23I). Passive force and total force were also significantly different between the groups (p=0.0027 and p=0.0014, respectively). Notably, such differences were not seen during recovery.

Interestingly, the opposite trend was observed for contraction active force and kinetics: no difference between the low-MFI and high-MFI groups during the stress phase, and significant differences during the recovery phase. These findings could indicate specific short-term and long-term effects on different aspects of cardiac contractility by autoantibodies present in the high MFI patients (FIG. 23J). Overall, the collected data suggest strong associations between cardiac stress, SLE-myocarditis autoantibody binding levels, and the severity of myocardial inflammation.

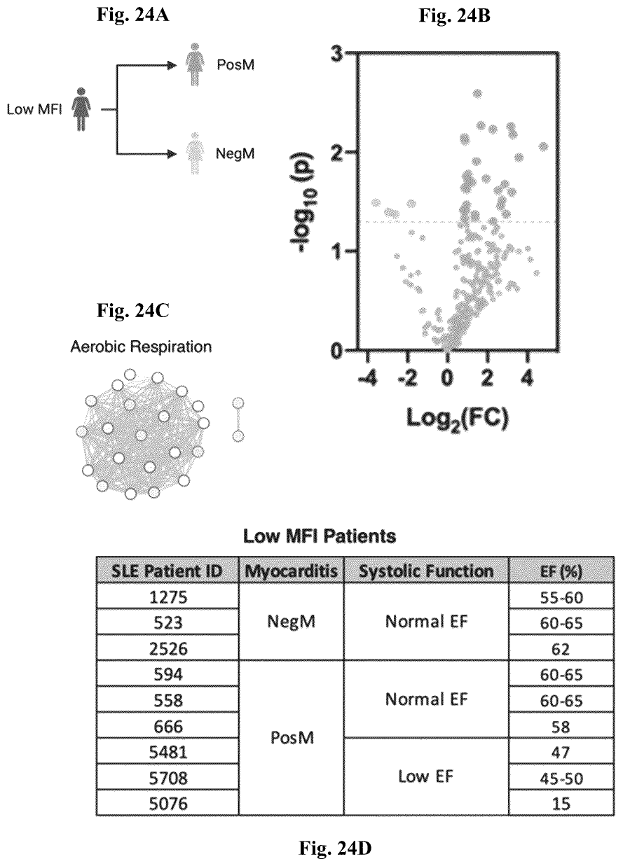

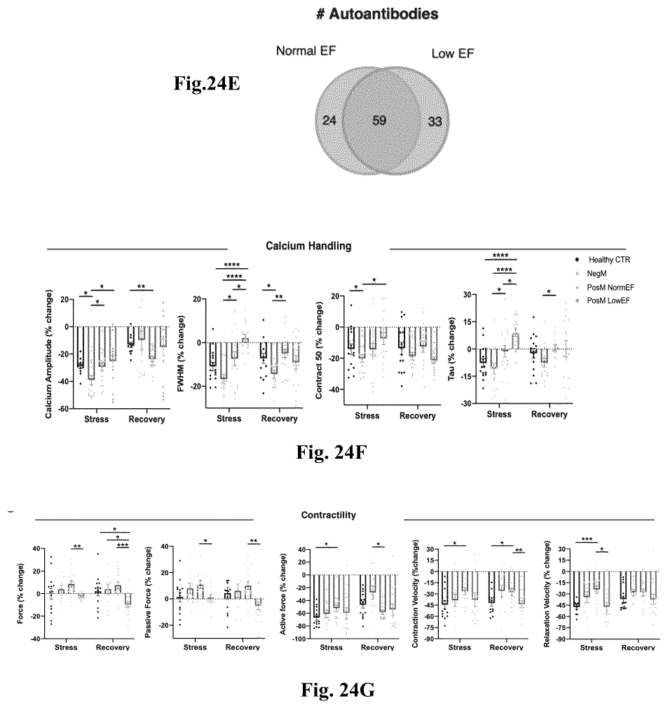

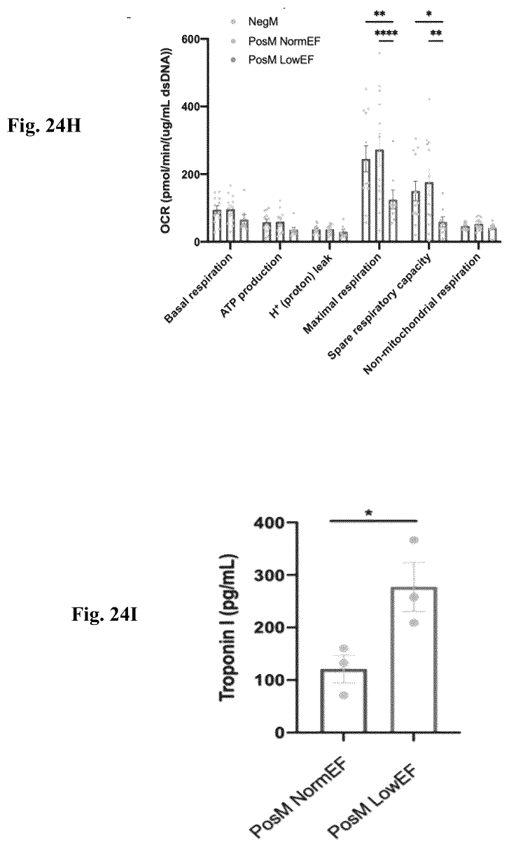

FIGS. 24A-I show that aspects of| linical subclassification of patients with low MFI revealed that distinct autoantibody profiles within each group resulted in differential effects on cardiac tissue performance. FIG. 24A shows SLE low MFI patient subclassification according to myocarditis diagnosis. FIG. 24B shows Volcano plots demonstrating differentially targeted autoantigens comparing SLE low MFI patients with myocarditis (right) to SLE low MFI patients without myocarditis and healthy controls (left), determined on the basis of p≤0.05 and log2(fold change) ≥1 (PosM: n=6 patients, NegM: n=3 patients, two-sided Student's t-tests). FIG. 24C shows that GO analysis revealed that autoantigens targeted by autoantibodies present in SLE low MFI patients with myocarditis are related to aerobic respiration.

FIG. 24D shows SLE low MFI patients with myocarditis according to their systolic function. FIG. 24E shows a Venn diagram representing the numbers of autoantigens identified in SLE low MFI myocarditis patients with normal EF and low EF. FIGS. 24F-G shows that calcium and brightfield imaging revealed that tissues cultured with autoantibodies from patients in different subclassification groups have distinct calcium handling properties (24F) and contractile behaviors (24G) (Healthy Control: n=16 tissues, NegM: n=15 tissues, Pos M Norm EF: n=18 tissues, PosM Low EF: n=16 tissues; two-sided Student's t-tests. FIG. 24H shows that real-time oxygen consumption rate measurements revealed that cardiomyocytes cultured with autoantibodies purified from patients in different subclassification groups demonstrated that autoantibodies present in myocarditis patients with low EF were associated with impaired respiration (NegM: n=12 iCM samples, Pos M Norm EF: n=12, PosM Low EF: n=8; two-way ANOVA with Tukey's multiple comparisons test). FIG. 24I shows that supernatant analysis demonstrated enhanced tissue troponin release in low EF patients when compared to normal EF patients, with and without myocarditis (PosM Norm EF: n=3, NegM Low EF: n=3; two-sided Student's t-test). Data are presented as mean ±. SEM. *p<0.05, **p<0.005, ***p<0.0005, ****p<0.0001.

Distinct autoantibody profiles among SLE patients with low binding levels resulted in differential functional tissue outcomes. Next, we investigated whether specific autoantibody profiles can impact cardiac function independently from overall levels of autoantibody binding to tissues. Such finding would suggest that specific autoantibody-autoantigen interactions can drive functional changes through mechanisms more subtle than sheer levels of autoantibody binding alone. Therefore, we focused on the myocarditis and non-myocarditis SLE patients with similarly low levels of autoantibody binding (low MFI) and assessed whether the antibodies in the myocarditis group recognized different autoantigens and had differential effects on cardiac tissues compared to non-myocarditis SLE patients (FIG. 24A). We found that 30 autoantigens were significantly more targeted by the autoantibodies from the patients with myocarditis, and only 4 autoantigens were significantly more targeted by the antibodies from patients without myocarditis (FIG. 24B). GO analysis followed by enrichment visualization revealed that the majority of autoantigens targeted by autoantibodies from low-MFI patients with myocarditis were associated with cellular respiration (FIG. 24C), suggesting that the heart muscle is at risk in these patients because of its high metabolic demand and constant need for ATP.

We further subclassified the low-MFI patients with myocarditis according to their systolic function (normal or low EF) to investigate whether patients with low EF have autoantibody profiles associated with more severe cardiac disease phenotype (FIG. 24D). The low-EF and normal-EF patients had 33 and 24 autoantibodies, respectively, that were unique for each group (FIG. 24E), while these groups combined had 59 autoantibodies that targeted the same cardiac autoantigens. Calcium transients in tissues cultured with autoantibodies from healthy controls and SLE patients with normal EF (with or without myocarditis) were shorter compared to baseline, while tissues cultured with autoantibodies from SLE-Myocarditis patients with low EF showed longer FWHM and τ decay during the stress phase, and no effect during recovery phase (FIG. 24F). Importantly, tissues cultured with autoantibodies purified from SLE-myocarditis patients with low EF had significantly lower total force and passive force compared to myocarditis patients with normal EF. These differences increased during the recovery phase. Finally, no difference in active contraction force or contraction kinetics was observed between the groups (FIG. 24G).

Since autoantigens targeted by autoantibodies isolated from the SLE-myocarditis patients were associated with pathways related to aerobic respiration, we asked whether they have an effect on mitochondrial function and ATP production. To probe this, we measured oxygen consumption rate (OCR) in cardiomyocytes cultured with patient's autoantibodies. While we identified autoantigens related to metabolism that were targeted by autoantibodies from SLE patients both with and without low EF, iPSC-cardiomyocytes cultured with autoantibodies isolated from SLE-myocarditis patients with low EF showed an interesting outcome. These iPSC-cardiomyocytes exhibited significantly lower spare respiratory capacity and maximum respiration rate than those cultured with autoantibodies isolated from SLE patients without myocarditis (p=0.01 and p=0.04, respectively) and SLE-myocarditis patients with normal EF (p=0.04), indicating impaired aerobic respiration and ATP production rates (FIG. 24H). Tissues cultured with autoantibodies from SLE-myocarditis patients with low EF had significantly higher levels of troponin in supernatant when compared to tissues cultured with autoantibodies from patients with normal EF, suggesting higher levels of cardiac injury (FIG. 24I).

These data indicate that the severity of cardiac disease (EF) correlates with the impact of autoantibodies on tissue function in vitro, independent from the level of autoantibody binding to tissues (MFI), implying that there are likely antibodies driving functional changes that are present in severe disease patients but not in those with less severe phenotype. Thus, the identification of such interactions could lead to the development of disease modifying therapeutics.

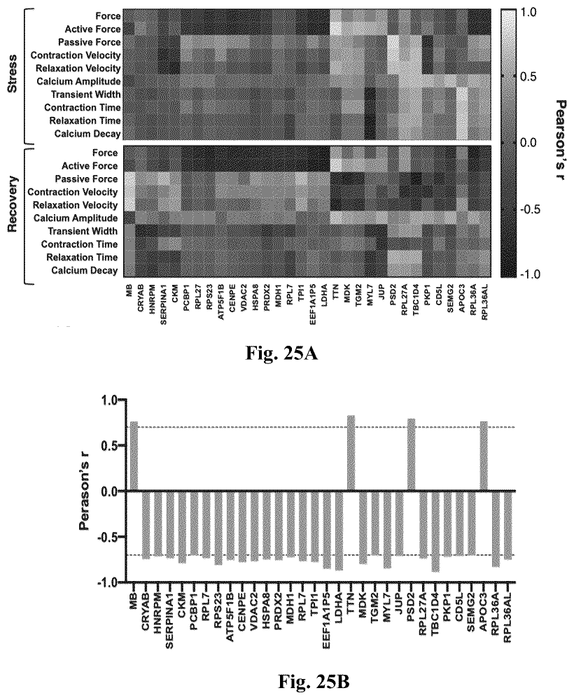

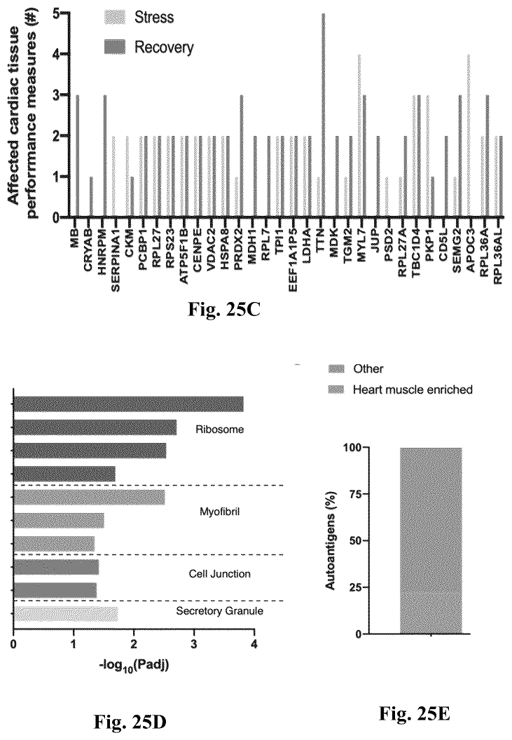

FIGS. 25A-E show that engineered cardiac tissue model enabled identifying candidate autoantibodies that directly alter cardiac tissue performance. FIG. 25A shows heat maps representing coefficients between in vitro cardiac tissue performance measures and autoantibody target quantifications. Candidate autoantibody targets were selected and presented in heatmaps based on at least one correlation with r>0.7 or r<−0.7. FIG. 25B shows that quantification of Pearson's r for each autoantibody target showed strongest correlation with an in vitro measure. FIG. 25C shows quantification of the number of correlations to in vitro measures with p<0.05 for each autoantibody target. FIG. 25D shows that top cellular compartment GO terms associated with candidate autoantibodies were related to ribosomes, myofibrils, cell junctions and secretory granules. FIG. 25E shows quantification of autoantigen cellular specificity.

The abundance of specific autoantibodies correlated with in vitro tissue function. To further investigate the specific autoantibodies driving functional changes in tissues, we evaluated the strength of correlations between the abundance of autoantibodies in patient serum as measured by LC-MS and the function of tissues treated with autoantibodies from the same patient and measured in vitro. A set of candidate autoantigens was selected based on Pearson correlations coefficients (>0.7 or <−0.7) with one or more in vitro metrics during the stress or recovery phases. The set was reduced to 32 autoantibodies identified in all samples from patients in the low MFI group with concomitant clinical myocarditis and decreased LVEF (FIGS. 25A, 25B).

Many autoantibodies showed distinct correlation patterns between the stress and recovery phases (FIG. 25A) with different numbers of strongly correlated variables (FIG. 25C), suggesting that the role of autoantibodies in driving tissue dysfunction may change over time and in response to stress. GO analysis of the identified autoantigens revealed significant associations with ribosomes, myofibrils, cell junctions, and secretory granules (FIG. 25D). Out of 32 autoantigens, seven were enriched in the heart muscle, making them strong candidates for further investigation as effectors of cardiac tissue dysfunction and potential therapeutic targets (FIG. 25E).

SLE patients have markedly higher rates of cardiovascular morbidity and mortality than the general population, largely due to myocarditis. The mechanisms of pathogenesis and the roles of autoantibodies involved in SLE-myocarditis are understudied, at least in part due the lack of controllable models capable of emulating the SLE phenotypes. Animal models are not predictive of autoantibody interactions with the adult human heart, due to inherent species-specific differences in cardiac physiology, while human cell cultures fail to recapitulate the complexity and function of the human heart.

We established an in vitro model in which cardiac tissues engineered from human iPS cells were incubated with the autoantibodies isolated from blood serum of SLA patients to study their role in the pathogenesis of autoimmune myocarditis in a physiologically relevant manner. This robust and technically simple model was established by studying a cohort of SLE patients and healthy controls following extensive clinical cardiovascular phenotyping (FIG. 21). The patients' autoantibodies were characterized for their distinct profiles by LC-MS, their binding to cardiac tissue antigens, and the resulting effects on tissue function (FIG. 20).

Functional tissue responses measured in vitro were correlated to clinical measures of SLE severity, myocarditis, and systolic dysfunction. Akin to how genetic variability impacts the disease pathogenesis, we show that distinct autoantibody patterns play a role in determining the heterogeneity of SLE-myocarditis using an engineered human cardiac tissue model. This unique model captures the heterogeneity of phenotypes of SLE myocarditis in a patient-specific manner and may help discover key etiologic factors and therapeutic targets of SLE-myocarditis.

While it is known that damage to fetal myocardium is associated with maternal autoantibodies to SSA/Ro and SSB/La, the role of autoantibodies in SLE-myocarditis in adults remains to be defined. It has been suggested that myocarditis develops following prolonged cellular stress in the myocardium that modifies the immunological presentation of cardiomyocytes. Electrical pacing has been shown to lead to oxidative stress and inflammatory gene expression, and we hypothesized that differential autoantigens presented in the heart following stress will lead to enhanced autoantibody binding and modulation of myocarditis and tissue dysfunction. Therefore, we induced the conditions of stress by stimulating engineered tissues at supraphysiological frequencies, enabling us to capture additional aspects of the disease in adults that might contribute to autoantibody-mediated myocarditis.

Another advantage of our platform is that it allows continuous and non-invasive measurements of cardiac tissue function before, during and after the induction of stress. We found that autoantibodies had diverging effects on cardiac tissue functionality during the stress and recovery phases. These findings mirror clinical observations in which organ damage is triggered during a SLE flare, but the damage persists following resolution of the inciting flare.

Furthermore, we found a striking correlation between patient myocarditis severity and the binding levels of autoantibodies to stressed cardiomyocytes. GO analysis confirmed that distinct autoantibodies in these individuals are associated with the contractile machinery and apoptosis in cardiomyocytes. These findings are in line with previous studies demonstrating that SLE autoantibodies bind to apoptotic fetal cardiomyocytes and apoptotic keratinocytes after prolonged damaging UV irradiation. Moreover, we report previously unknown associations between cardiac stress, autoantibody binding profiles, and the severity of SLE-myocarditis.

Currently, SLE-myocarditis is clinically diagnosed by transthoracic echocardiograms (TTE) and electrocardiograms (ECG) that can identify cardiac abnormalities such as reduced EF, non-specific ST changes, sinus tachycardia, and long QTC. However, these cardiac abnormalities are not specific for myocarditis and many SLE-myocarditis patients remain undiagnosed and therefore untreated. Our group has previously reported that FDG-PET can be used to diagnose SLE-myocarditis, even in patients without any clinical cardiac abnormalities. In this study, we demonstrated that autoantibody binding levels to stressed cardiac tissues had stronger correlations to myocarditis severity than any other clinical measure in the entre cohort of patients. We have also shown that these autoantibodies altered both calcium handling and contractility of the ECTs. These finding indicate the potential of our platform to be used as an accessible methodology to diagnose subclinical patients with severe myocarditis. Future studies are required to determine whether high binding levels also lead to enhanced immune cell infiltration and contribute to increased inflammation.

Additionally, we found that even when controlling for the levels of binding to cardiac tissues, distinct autoantibody patterns existed between patients with and without myocarditis, indicating that not only reactivity levels, but also the specific autoantibody patterns lead to myocarditis in certain individuals. Using LC-MS we were able to identify that SLE-myocarditis patients have autoantibodies targeting autoantigen populations that play a role in aerobic respiration. The heart is an organ with extremely high metabolic demand, making it highly susceptible to risk if targeted by autoantibodies with reactivities to antigens associated with ATP production. An important feature of the model is the switch from medium promoting glycolysis to medium supporting fatty acid oxidation and aerobic respiration, making it suitable to study the effect of those autoantibodies on cardiac tissue function.

Clinical data revealed heterogeneous manifestations of SLE-myocarditis, with a range of EF, from normal to severely decreased. The model was sensitive enough to capture this heterogeneity and to attribute these functional differences, at least in part, to differences in autoantibody profiles. Tissues treated with autoantibodies collected from patients with low EF demonstrated impaired calcium handling, force generation and respiration when compared to those treated with autoantibodies from patients with normal EF and SLE patients with no myocarditis. While most autoantibodies between patients with low EF and normal EF overlapped, 25-30% were distinct, suggesting their role in the heterogeneity of disease mechanisms. In patients with low EF and tissue function measures, we identified autoantibodies that exert functional effects in vitro and warrant further study of their potential impacts in vivo.

We propose that the cardiac tissue model reported here could be used for the discovery and validation of autoantibodies that aggravate SLE disease, an application not previously reported for engineered tissue models. The model could also be utilized to validate the potential of blocking such autoantibodies with cognate antigens as a therapeutic approach.

The autoantibody responses in vitro are measured without the immune cell components, thus not capturing the complexity of the immune response in SLE-myocarditis. Furthermore, studying interactions of autoantibodies with cardiac tissues generated from iPS cells enables robustness and scalability, however it does not capture genetic variations between SLE-myocarditis patients. Additional studies are needed to elucidate any effects of genetic factors. The present study may also be underpowered for detecting subtle differences in autoantibodies between patient samples, which result from the heterogeneity of SLE patient autoantibody profiles. Future studies using the proposed methodology should include larger patient cohorts for enhanced autoantibody detection.

In conclusion, the use of engineered cardiac tissue models holds great promise for elucidating autoimmune mediated myocarditis disease mechanisms and identifying disease-aggravating autoantibodies. This patient-specific system captures the individual susceptibility to myocardial dysfunction and can help develop and test new therapeutic strategies. This approach could be readily extended to studies of other autoimmune diseases with myocardial tissue damage, such as rheumatoid arthritis, and enable revealing of diverse landscapes of disease that affect the human heart.

### Methods and Materials

### Patients

All study participants signed an informed consent form. The study was approved by the Columbia University Institutional Review Board.

Patients were recruited from the Columbia University Lupus Cohort. Patients were 18 years of age or older and met the 1997 American College of Rheumatology (ACR) classification criteria for SLE. Exclusion criteria included a known prior diagnosis of cardiovascular disease (defined as self-reported or physician-diagnosed MI, heart failure, coronary artery revascularisation, angioplasty, peripheral vascular disease, implanted pacemaker or defibrillator devices and atrial fibrillation); or a diagnosis of known myocarditis, or major ST-T changes prior to enrollment. These inclusion/exclusion criteria aimed to reflect an active myocardial process and avoid a selection bias of chronic condition.

Ninety-two percent of the patients were female. Sixty-nine percent of the study participants were Hispanic, 25% were white, and 6% were black. The mean age of the patients was 34 years, and mean disease duration was 13 years. Half of the patients had lupus nephritis and 8% had antiphospholipid antibody syndrome. Eighty-three percent were on antimalarials, 45% were on glucocorticoids, and 75% were on immunosuppressants.

### Clinical Covariates

Demographics, comorbidities, and smoking history were self-reported and collected from questionnaires and patient interviews. Hypertension was defined as a systolic BP of ≥140 mm Hg, diastolic BP of ≥90 mm Hg at the time of the evaluation or antihypertensive medication use. Diabetes was defined as glycated hemoglobin (HbA1c) greater than 6.4% or use of diabetes medications. All medications were documented from patient interview and medical records. SLE disease duration was defined as the duration in years from the date of physician diagnosis. SLE disease activity was calculated using the Systemic Lupus Erythematosus Disease Activity Index 2000 (SLEDAI-2K).

### Laboratory Covariates

Autoantibodies including antinuclear antibodies, anti-SSA/Ro, anti-SSB/La, anti-ds-DNA, anti- Smith, anti-RNP, antiphospholipid antibodies and other pertinent laboratories such as complement levels (C3, C4), erythrocyte sedimentation rate (ESR), high-sensitivity C reactive protein, troponin and pro-beta-natriuretic peptide (pro-BNP) levels, were assessed at the clinical laboratory at New York Presbyterian Hospital and the Core Laboratory of the Columbia University Irving Institute for Clinical and Translation Research.

### ¹⁸F-FDG PET/CT

¹⁸F-FDG PET/CT Myocardial uptake imaging was performed on an MCT 64 PET/CT scanner (Siemens Medical Solutions USA, Knoxville, Tennessee, USA). A low-dose CT transmission scan (120 kV, 25 mA) was obtained for attenuation correction of PET data. All patients were on a carbohydrate-free diet for 24 hours. Patients were injected with 10±0.1 mCi of ¹⁸F-FDG intravenously using an antecubital or dorsal forearm catheter. A list mode 3D PET scan was acquired for 10 min following a 90 min uptake period post-F-FDG injection. Non-gated attenuation-corrected images were reconstructed yielding 3 mm effective resolution. Corridor 4DM software was used to visually assess myocardial ¹⁸F-FDG uptake as well as semi-automatically quantify mean radiotracer uptake in the myocardium. Quantification of inflammation by ¹⁸F-FDG PET/CT involved measurement of SUV in the myocardium.

### Antigen Enrichment for LC-MS

Immunoprecipitations were performed by cross-linking 0.5 μL of patient serum to 25 μL of protein A/G beads (Thermo Fisher Scientific). Beads were subsequently washed with 2 mL of washbuffer, and disuccinimidyl suberate (1XDSS) was used to cross-link the autoantibodies to the beads. A glycine buffer (pH=3) was then used to wash the cross-linked beads. Following, beads were incubated with 1 mg of cardiac tissue lysate for 1 hr. Beads were subsequently washed and eluted with an SDS/urea buffer (5% SDS, 8M urea, 100 mM glycine).

### Sample Preparation for Mass Spectrometry Analysis

The enriched antigens were reduced using dithiothreitol (5 μl of 0.2 M) for 1 h at 57° C. and subsequently alkylated with iodoacetamide (5 μl of 0.5 M) for 45 min in dark at room temperature. Samples were loaded onto S-Trap microcolumns (Protifi, USA) according to the manufacturer's instructions. Briefly, 3 μL of 12% phosphoric acid and 165 μL of binding buffer (90% methanol, 100-mM TEAB) were added to each sample. Samples were loaded onto the S-trap columns and centrifuged at 4000×g for 30 s. After three washes, 20 μL of 50-mM TEAB and 1 μg of trypsin (1:50 ratio) were added to the trap and incubated at 47° C. for one hour. Peptides were then eluted using 40% acetonitrile (ACN) in 0.5% acetic acid followed by 80% ACN in 0.5% acetic acid. Eluted peptides were dried and concentrated in a SpeedVac.

### LC-MS/MS Analysis

One tenth of each sample was loaded onto a trap column (Acclaim PepMap 100 pre-column, 75 μm×2 cm, C18, 3 μm, 100Å, Thermo Scientific) connected to an analytical column (EASY-Spray column, 50 μm×75 μm ID, PepMap RSLC C18, 2 μm, 100 Å, Thermo Scientific) using the autosampler of an Easy nLC 1000 (Thermo Scientific) with solvent A consisting of 2% acetonitrile in 0.5% acetic acid and solvent B consisting of 80% acetonitrile in 0.5% acetic acid. The peptide mixture was gradient-eluted into an Orbitrap Eclipse Tribrid mass spectrometer (Thermo Scientific) using the following gradient: 5%-15% solvent B over 60 min, 15%-25% solvent B over 35 min, 25%-40% solvent B over 20 min, and 40-100% solvent B over 10 min. The full scan was acquired from m/z 400-1500 with a resolution of 240,000 (@m/z 200), a target value of 10⁶ and a maximum ion time of 50 ms. The MS/MS spectra were collected in the ion trap with an AGC target of 2*10⁶, maximum ion time of 18 msec, one microscan, 0.7 m/z quadrupole isolation window, fixed first mass of 110 m/z, Normalized Collision Energy (NCE) of 27, and dynamic exclusion of 30 sec.

### LC-MS Data Analysis

All acquired MS2 spectra were searched against the UniProt human reference database using Andromeda within the MaxQuant search engine (Version 1.6.3.4) and the following search parameters: precursor mass tolerance ±10 ppm, fragment mass tolerance ±0.4 Da, digestion parameters trypsin allowing two missed cleavages, fixed modification of carbamidomethyl on cysteine, variable modification of oxidation on methionine, and deamidation on glutamine and asparagine. The results were filtered to <1% peptide and protein False Discovery Rate (FDR) searched against a decoy database and requiring proteins to have at least two unique peptides. For detection analysis, raw intensity values were used, and antigens were filtered based on detection in all samples from the group being analyzed. For intensity comparisons between groups, the raw intensities were log2 transformed and proteins further filtered for being present in at least 3 replicates of one sample group. Missing values were replaced from normal distribution in Perseus and a Student's t-test was applied.

Cardiomyocyte Derivation from Human iPSCs

To visualize calcium handling in real-time, WTC11-GCaMP6f iPSCs that contain a constitutively expressed GCaMP6f calcium-responsive fluorescent protein inserted into a single allele of the AAVS1 safe harbor locus were used. WTC11-GCaMP6f were obtained through material transfer agreements from B. Conklin, Gladstone Institutes. Cardiomyocytes were differentiated as previously described⁴⁶. On Day 10, RPMI-no glucose (Life Technologies, cat. no. 11879020) supplemented with B27 (Thermo Fisher Scientific, cat. no. 17504044) and 213 μg/mL ascorbic acid (Sigma-Aldrich, cat. no. A445), was used to purify the iPSC-CMs population and eliminate potential contaminating mesodermal and endodermal populations. Medium was replaced on day 13 with RPMI-B27 medium supplemented with 213 μg/ml ascorbic acid until day 16.

On day 17 cells were pretreated with rock-inhibitor (y-27632 dihydrochloride, 5 μM) for 4 hours before dissociation. Cells were dissociated by enzyme digestion with collagenase type II (95 U/mL; Worthington, cat. no. LS004176) and pancreatin (0.6 mg/mL; Sigma-Aldrich, cat. P7545) in dissociation buffer (Glucose (5.5 mM), CaCl₂·2H₂0 (1.8 mM), KCl (5.36 mM), MgSO₄·7H₂O (0.81 mM), NaCl (0.1M), NaHCO₃ (0.44 mM), NaH₂PO₄ (0.9 mM)) on a shaker in a 37° C. incubator. Flow cytometry for cTnT+(BD BioSciences cat. no 565744) was performed prior to cell use for tissue fabrication to ensure cell purity (>90% cTnT+).

### Cardiac Fibroblasts

Primary Human cardiac fibroblasts (NHCF-V; Lonza, cat. no. CC-2904) and dermal fibroblasts (NHDFs; Lonza, cat. no. CC-2509) were cultured according to the manufacturer's recommendations.

### Bioreactor Fabrication

The platform, termed milliPillar, was assembled as described herein. Briefly, the platform was fabricated by casting polydimethylsiloxane (PDMS) into custom molds containing carbon electrodes and was cured in an oven at 65° C. The platform was then detached from the mold and plasma bonded to a glass slide. After completion, each well contained a set of horizontal flexible pillars upon which engineered tissues can suspended, as described below. Each well also contained a set of carbon electrodes for electrical field stimulation. A custom Arduino-based electrical stimulator was used for electrical pacing during culture and video acquisition, as previously described.

### Engineering and Culture of Human Cardiac Tissues

Differentiated hiPS-CMs (75%) were mixed with supporting cardiac fibroblasts (25%) and resuspended in RPMI-B27 (RPMI 1640 basal medium, L-ascorbic acid 2-phosphate, bovine serum albumin BSA, and B27 supplement). The cells were resuspended in fibrinogen by mixing 33 mg/mL human fibrinogen (Sigma-Aldrich, cat. no. F3879) with the cell solution to a final fibrinogen concentration of 5 mg/mL and cell concentration of 370,000 cells/μL. An aliquot of 3 μL of thrombin (2 U/mL) were added to each well. A 12 μL aliquot of the cell solution was dispensed into each well of the platform and allowed to polymerize at 37° C. for 15 min, so that the tissues readily formed around the pillars. 400 μL of RPMI-B27 with 213 μg/mL ascorbic acid, 10 uM Rock inhibitor and 5 mg/mL 6-aminocaproic acid (Sigma-Aldrich, cat. no. A7824) were added to the wells. After 24 hr (day 1), medium was changed to MM medium that was formulated as previously described.

On day 2, platforms were randomized into two different culture medium groups; RPMI-B27 and MM. On day 7 electrical stimulation was initiated at a frequency of 2 Hz until day 14. Tissue functionality was assessed by calcium and brightfield imaging on day 14. Force generation, contraction and relaxation velocities, and calcium transient amplitude and kinetics were measured (FIG. 27). After evaluating tissue function under two different medium conditions, MM was chosen as the culture medium for subsequent experiments with patient autoantibodies.

Human Cardiac Tissue Incubation with Autoantibodies

Purified autoantibodies from SLE patients without (n=3) and with myocarditis (n=9), and from healthy controls (n=3) were purified from patient sera using NAb™ Protein A/G Spin Columns, (Thermo Fisher Scientific, cat. no. 89954). Autoantibody purification from the patients' sera was performed to eliminate confounding effects of sera components such as cytokines, growth factors and drugs. The purified autoantibodies were supplemented to the culture medium on day 14, with tissues stimulated at 2 Hz and maintained for an additional 14 days. During this time, the frequency of electrical stimulation was stepwise increased to 6 Hz by day 21 (stress phase) and decreased to 2 Hz thereafter and maintained until day 28 (recovery phase) (FIG. 25G). Furthermore, to exclude the possibility that the functional effect on cardiac tissue performance is not associated to titer levels and only to distinct autoantibody patterns, the same concentration of purified patient autoantibodies, 0.5 μg/mL was added to all tissues.

To assess the effect of purified autoantibodies on cardiac tissue functionality we evaluated the tissue contractility and calcium handling (the full list of metrics and their descriptions is provided in FIG. 27) at baseline (day 14), during the stress phase (day 21), and during the recovery phase (day 28). To reduce noise from tissue-to-tissue variability measurements were normalized to the baseline prior to the addition of autoantibodies.

### Immunostaining

Whole mount engineered cardiac tissues were fixed and permeabilized in 100% ice cold methanol for 10 min, washed three times in PBSX1, and then blocked for 1 hr at room temperature in PBSX1 with 2% fetal bovine serum (FBS). After blocking, the tissues were incubated with primary mouse anti-α-actinin (sarcomeric) antibody (1:750, Sigma-Aldrich, cat. no. A7811), and vimentin (Abcam, cat. no. ab24525) washed three times and incubated for 1 hr with secondary antibodies (Millipore Sigma, cat. no. AP194C; Thermo Fischer, cat. no. A-21206; Thermo Fischer, cat. no. A-31571). For nuclei detection, the tissues were washed and incubated with NucBlue (Thermo Fisher, cat. no. R37606). For indirect binding stain secondary anti-human IgG was used (Thermo Fisher, cat. no. A-11013). Samples were visualized using a scanning laser confocal microscope (Nikon Eclipse Ti).

### Calcium Imaging and Analysis

Tissues were imaged in a live-cell chamber (STX Temp & CO2 Stage Top Incubator, Tokai Hit) using a sCMOS camera (Zyla 4.2, Andor Technology) connected to an inverted fluorescence microscope with a standard GFP filter set (Olympus IX-81). Tissues were then electrically stimulated, and videos were acquired at 20 frames per second (fps) for 4600 frames to measure excitation threshold and maximum capture rate or 300 frames (stimulated at 1 Hz) to measure calcium flux as previously described. Calcium signals were analyzed from calcium imaging videos recorded at 20 fps. Briefly, a custom Python script was developed to average the pixel intensities for each frame, and this transient was then corrected for fluorescent decay. Further analysis by the script then extracted the metrics of calcium handling including calcium amplitude, full width half max (FWHM), contract 50, and the exponential decay constant (τ). A description of these metrics is provided in FIG. 27.

### Brightfield Imaging and Analysis

For force generation measurements, videos were then acquired at 20 fps for 4800 frames using a custom program to stimulate cardiac tissues from 0.5 Hz to 4 Hz as previously described, and force generation was analyzed from brightfield videos. Briefly, a custom Python script was developed to track the motion of the pillar heads and to calculate the force by multiplying the displacement of the pillars with the coefficient determined from the force-displacement calibration curve generated for the pillars. The script used the location of the pillar heads to determine the total deflection of the pillar from their equilibrium position without any force applied. Further analysis of the deflection trace extracted metrics of contractility, including the following: force, passive force, active force, contraction velocity, and relaxation velocity.

### Analysis of Tissue Functional Metrics

Following feature extraction, tissues that did not capture at 1 Hz stimulation based on both calcium and contractility analysis at baseline were removed from further analysis. The data was then processed to remove outliers greater than or less than +/−1.5 times the interquartile range for each group. Outliers were removed from each metric independently. The metrics for each tissue were internally normalized to baseline values and presented as percent change from baseline. For correlation analyses, Pearson's correlation coefficients were calculated and reported. Statistics were preformed using the SciPy and scikit-learn Python libraries.

### Metabolic Analysis

The Seahorse XF96 analyzer was used to evaluate mitochondrial function. Prior to the assay, cells were cultured with antibodies for 5 days. Two days prior to the assay, the cardiomyocytes were dissociated using TrypLE Select Enzyme (10X) and replated onto an XF96 cell culture microplate coated with Matrigel at a cell density of 80,000 per XF96 well. For the remaining two days, the cells continued to be cultured in media treated with antibodies.

To perform the Agilent Seahorse XF Cell Mito Stress Test, 1 hour prior to the assay, the culture medium was exchanged for assay medium (XF RPMI Medium, pH 7.4 supplemented with 1 mM pyruvate, 2 mM glutamine, and 10 mM, glucose) and the culture plate was placed in a non-CO₂incubator. During the Mito Stress Test, electron transport chain modulators Oligomycin (1.5 uM), FCCP (0.5 uM), and Rotenone and antimycin A (0.5 uM) were injected serially into each well. The oxygen consumption rate (OCR) values were normalized to the average concentration of dsDNA present in each well of the plate, which were determined using the Quanti-iT PicoGreen dsDNA Assay Kit (Thermo Fisher cat. no. P7589). The key parameters of mitochondrial function determined by the Mito Stress Test, including basal respiration, ATP-linked respiration, proton leak, maximal respiration, spare capacity, and non-mitochondrial oxygen consumption were generated using the Seahorse XF Mito Stress Test Report Generator.

1. A system to culture human cardiac muscle tissues comprising a bioreactor comprising a plurality of culture wells arranged linearly between two carbon electrodes that are exposed within each well; and
two horizontal parallel flexible pillars extending from each well wherein the pillars in each well are configured to suspend an engineered tissue
and comprising a double bi-layer capacitor comprising apart carbon rods for reproducibly injecting a safe amount of charge that is compatible with cardiac tissue stimulation.

2. The system of claim 1 further comprising circuitry and software to provide cyclic electromechanical stimulation to the cardiac muscle tissues.

3. The system of claim 1 further comprising a microscope and software to control and synchronize video acquisition, electrical stimulation, and stage positioning.

4. The system of claim 3 wherein brightfield imaging and calcium imaging are acquired.

5. The system of claim 1 wherein the engineered human cardiac muscle tissues are generated from induced pluripotent stem cell (iPSC)-derived cardiomyocytes, human dermal fibroblasts, cardiac fibroblast, and tissues from iPS-cardionyocytes and iPS-cardiac fibroblasts.

6. The system of claim 1 wherein the engineered human cardiac muscle tissues comprise a collagen or fibrin hydrogel.

7. The system of claim 1 wherein the engineered human cardiac muscle tissues are derived from induced pluripotent stem cell (iPSC)-derived cardiomyocytes and cardiac fibroblasts within a fibrin hydrogel.

8. A method for culturing engineered human cardiac muscle tissues, the method comprising disposing the tissues between the two horizontal parallel flexible pillars of the reactor of claim 1; and
providing cyclic electromechanical stimulation to the tissues.

9. The method of claim 8 wherein the engineered human cardiac muscle tissues are generated from induced pluripotent stem cell (iPSC)-derived cardiomyocytes, human dermal fibroblasts, cardiac fibroblast, and tissues from iPS-cardionyocytes and iPS-cardiac fibroblasts.

10. The method of claim 8 wherein the engineered human cardiac muscle tissues comprise a collagen or fibrin hydrogel.

11. The method of claim 8 wherein the tissues are derived from induced pluripotent stem cell (iPSC)-derived cardiomyocytes and cardiac fibroblasts within a fibrin hydrogel.

12. A method for diagnosing myocarditis in a subject, the method comprising:
culturing engineered cardiac muscle tissues in the system of claim 1;
adding the subject's blood serum or isolated antibodies to the reactor;
culturing the engineered cardiac muscle tissues in the presence of the blood serum or antibodies for a period of time;
measuring calcium handling and force generation of the engineered cardiac muscle tissue or immunostaining the tissues; and determining whether the measured calcium handling or force generation or immunostaining indicate that the subject is suffering from myocarditis.

13. The method of claim 12 wherein culturing engineered cardiac muscle tissues comprises disposing the tissues between the two horizontal parallel flexible pillars of the reactor; and
providing cyclic electromechanical stimulation to the tissues.

14. The method of claim 12 wherein the subject is suffering from systemic lupus erythematosus or rheumatoid arthritis.

15. The method of claim 12 wherein the engineered human cardiac muscle tissues are generated from induced pluripotent stem cell (iPSC)-derived cardiomyocytes, human dermal fibroblasts, cardiac fibroblast, and tissues from iPS-cardionyocytes and iPS-cardiac fibroblasts.

16. The method of claim 12 wherein the engineered human cardiac muscle tissues comprise a collagen or fibrin hydrogel.

17. The method of claim 12 wherein the engineered human cardiac muscle tissues are derived from induced pluripotent stem cell (iPSC)-derived cardiomyocytes and cardiac fibroblasts within a fibrin hydrogel.

18. The method of claim 12 wherein the engineered human cardiac muscle tissues are co-cultured with autoantibodies from subjects' sera.

19. The method of claim 12 wherein ¹⁸F-fluorodeoxyglucose-positron emission tomography/computed tomography (¹⁸F-FDG-PET/CT) is used to quantify extent of myocardial inflammation.

20. The method of claim 12 comprising culturing the engineered human cardiac muscle tissues in a medium to shift cell metabolism from anaerobic glycolysis toward fatty acid oxidation; and
subjecting the engineered human cardiac muscle tissues to electrical stimulation to induce macroscopic contractions.
